SEQUENCE LISTING (201 sequences)

SEQ ID NO: 1 - protein, 5 aa, Artificial Sequence
  5-mer Peptoprime
WMWCW

SEQ ID NO: 2 - protein, 5 aa, artificial sequence
  5-mer Peptoprime
MWCWM

SEQ ID NO: 3 - protein, 5 aa, Artificial Sequence
  5-mer Peptoprime
MWCWY

SEQ ID NO: 4 - protein, 5 aa, Artificial Sequence
  5-mer Peptoprime
WMWCN

SEQ ID NO: 5 - protein, 5 aa, Artificial Sequence
  5-mer Peptoprime
WMWCE

SEQ ID NO: 6 - protein, 5 aa, Artificial Sequence
  5-mer Peptoprime
MWCWT

SEQ ID NO: 7 - protein, 5 aa, Artificial Sequence
  5-mer Peptoprime
MWMWC

SEQ ID NO: 8 - protein, 5 aa, Artificial Sequence
  5-mer Peptoprime
YWMWC

SEQ ID NO: 9 - protein, 5 aa, Artificial Sequence
  5-mer Peptoprime
NWMWC

SEQ ID NO: 10 - protein, 5 aa, Artificial Sequence
  5-mer Peptoprime
MWCWQ

SEQ ID NO: 11 - protein, 5 aa, Artificial Sequence
  5-mer Peptoprime
WMCYH

SEQ ID NO: 12 - protein, 5 aa, Artificial Sequence
  5-mer Peptoprime
WMCQY

SEQ ID NO: 13 - protein, 5 aa, Artificial Sequence
  5-mer Peptoprime
MWMCT

SEQ ID NO: 14 - protein, 5 aa, Artificial Sequence
  5-mer Peptoprime
HFWMC

SEQ ID NO: 15 - protein, 5 aa, Artificial Sequence
  5-mer Peptoprime
WYWMC

SEQ ID NO: 16 - protein, 5 aa, Artificial Sequence
  5-mer Peptoprime
QNWMC

SEQ ID NO: 17 - protein, 5 aa, Artificial Sequence
  5-mer Peptoprime
MQWMC

SEQ ID NO: 18 - protein, 5 aa, Artificial Sequence
  5-mer Peptoprime
DQWMC

SEQ ID NO: 19 - protein, 5 aa, Artificial Sequence
  5-mer Peptoprime
WMCHI

SEQ ID NO: 20 - protein, 5 aa, Artificial Sequence
  5-mer Peptoprime
WMCSW

SEQ ID NO: 21 - protein, 5 aa, Artificial Sequence
  5-mer Peptoprime
CECWF

SEQ ID NO: 22 - protein, 5 aa, Artificial Sequence
  5-mer Peptoprime
WDCWF

SEQ ID NO: 23 - protein, 5 aa, Artificial Sequence
  5-mer Peptoprime
MWCHM

SEQ ID NO: 24 - protein, 5 aa, Artificial Sequence
  5-mer Peptoprime
WCQHM

SEQ ID NO: 25 - protein, 5 aa, Artificial Sequence
  5-mer Peptoprime
DCWHM

SEQ ID NO: 26 - protein, 5 aa, Artificial Sequence
  5-mer Peptoprime
WCPCM

SEQ ID NO: 27 - protein, 5 aa, Artificial Sequence
  5-mer Peptoprime
WWYCM

SEQ ID NO: 28 - protein, 5 aa, Artificial Sequence
  5-mer Peptoprime
WHHCM

SEQ ID NO: 29 - protein, 5 aa, Artificial Sequence
  5-mer Peptoprime
WMCCM

SEQ ID NO: 30 - protein, 5 aa, Artificial Sequence
  5-mer Peptoprime
QWCCM

SEQ ID NO: 31 - protein, 5 aa, Artificial Sequence
  5-mer Peptoprime
WWQCM

SEQ ID NO: 32 - protein, 5 aa, Artificial Sequence
  5-mer Peptoprime
MFWCM

SEQ ID NO: 33 - protein, 5 aa, Artificial Sequence
  5-mer Peptoprime
NWWCM

SEQ ID NO: 34 - protein, 5 aa, Artificial Sequence
  5-mer Peptoprime
MCHWM

SEQ ID NO: 35 - protein, 5 aa, Artificial Sequence
  5-mer Peptoprime
CMCWM

SEQ ID NO: 36 - protein, 5 aa, Artificial Sequence
  5-mer Peptoprime
HWCWM

SEQ ID NO: 37 - protein, 5 aa, Artificial Sequence
  5-mer Peptoprime
HMWWM

SEQ ID NO: 38 - protein, 5 aa, Artificial Sequence
  5-mer Peptoprime
MWHWP

SEQ ID NO: 39 - protein, 5 aa, Artificial Sequence
  5-mer Peptoprime
NWCWP

SEQ ID NO: 40 - protein, 5 aa, Artificial Sequence
  5-mer Peptoprime
WWIMY

SEQ ID NO: 41 - protein, 5 aa, Artificial Sequence
  5-mer Peptoprime
MWCHY

SEQ ID NO: 42 - protein, 5 aa, Artificial Sequence
  5-mer Peptoprime
CWMWY

SEQ ID NO: 43 - protein, 5 aa, Artificial Sequence
  5-mer Peptoprime
WCTWY

SEQ ID NO: 44 - protein, 5 aa, Artificial Sequence
  5-mer Peptoprime
MCHWY

SEQ ID NO: 45 - protein, 5 aa, Artificial Sequence
  5-mer Peptoprime
WHCWY

SEQ ID NO: 46 - protein, 5 aa, Artificial Sequence
  5-mer Peptoprime
CNWWY

SEQ ID NO: 47 - protein, 5 aa, Artificial Sequence
  5-mer Peptoprime
MCWWY

SEQ ID NO: 48 - protein, 5 aa, Artificial Sequence
  5-mer Peptoprime
MCMCN

SEQ ID NO: 49 - protein, 5 aa, Artificial Sequence
  5-mer Peptoprime
WCMWE

SEQ ID NO: 50 - protein, 5 aa, Artificial Sequence
  5-mer Peptoprime
WCMWV

SEQ ID NO: 51 - protein, 5 aa, Artificial Sequence
  5-mer Peptoprime
HCWHT

SEQ ID NO: 52 - protein, 5 aa, Artificial Sequence
  5-mer Peptoprime
MWHCT

SEQ ID NO: 53 - protein, 5 aa, Artificial Sequence
  5-mer Peptoprime
MWWCT

SEQ ID NO: 54 - protein, 5 aa, Artificial Sequence
  5-mer Peptoprime
WWCQT

SEQ ID NO: 55 - protein, 5 aa, Artificial Sequence
  5-mer Peptoprime
NWCMH

SEQ ID NO: 56 - protein, 5 aa, Artificial Sequence
  5-mer Peptoprime
CMWPH

SEQ ID NO: 57 - protein, 5 aa, Artificial Sequence
  5-mer Peptoprime
CMWHH

SEQ ID NO: 58 - protein, 5 aa, Artificial Sequence
  5-mer Peptoprime
WYMCH

SEQ ID NO: 59 - protein, 5 aa, Artificial Sequence
  5-mer Peptoprime
MCWDH

SEQ ID NO: 60 - protein, 5 aa, Artificial Sequence
  5-mer Peptoprime
HHMWH

SEQ ID NO: 61 - protein, 5 aa, Artificial Sequence
  5-mer Peptoprime
CWMWH

SEQ ID NO: 62 - protein, 5 aa, Artificial Sequence
  5-mer Peptoprime
TCWWH

SEQ ID NO: 63 - protein, 5 aa, Artificial Sequence
  5-mer Peptoprime
WCWWH

SEQ ID NO: 64 - protein, 5 aa, Artificial Sequence
  5-mer Peptoprime
WCMMC

SEQ ID NO: 65 - protein, 5 aa, Artificial Sequence
  5-mer Peptoprime
MWYMC

SEQ ID NO: 66 - protein, 5 aa, Artificial Sequence
  5-mer Peptoprime
MWHMC

SEQ ID NO: 67 - protein, 5 aa, Artificial Sequence
  5-mer Peptoprime
EWCMC

SEQ ID NO: 68 - protein, 5 aa, Artificial Sequence
  5-mer Peptoprime
WWCMC

SEQ ID NO: 69 - protein, 5 aa, Artificial Sequence
  5-mer Peptoprime
WHWMC

SEQ ID NO: 70 - protein, 5 aa, Artificial Sequence
  5-mer Peptoprime
WYNPC

SEQ ID NO: 71 - protein, 5 aa, Artificial Sequence
  5-mer Peptoprime
WWHPC

SEQ ID NO: 72 - protein, 5 aa, Artificial Sequence
  5-mer Peptoprime
MMWYC

SEQ ID NO: 73 - protein, 5 aa, Artificial Sequence
  5-mer Peptoprime
CWMHC

SEQ ID NO: 74 - protein, 5 aa, Artificial Sequence
  5-mer Peptoprime
WEYHC

SEQ ID NO: 75 - protein, 5 aa, Artificial Sequence
  5-mer Peptoprime
WWCKC

SEQ ID NO: 76 - protein, 5 aa, Artificial Sequence
  5-mer Peptoprime
CWWKC

SEQ ID NO: 77 - protein, 5 aa, Artificial Sequence
  5-mer Peptoprime
WMYCC

SEQ ID NO: 78 - protein, 5 aa, Artificial Sequence
  5-mer Peptoprime
MWYCC

SEQ ID NO: 79 - protein, 5 aa, Artificial Sequence
  5-mer Peptoprime
HDWCC

SEQ ID NO: 80 - protein, 5 aa, Artificial Sequence
  5-mer Peptoprime
CWWQC

SEQ ID NO: 81 - protein, 5 aa, Artificial Sequence
  5-mer Peptoprime
HYMWC

SEQ ID NO: 82 - protein, 5 aa, Artificial Sequence
  5-mer Peptoprime
YHMWC

SEQ ID NO: 83 - protein, 5 aa, Artificial Sequence
  5-mer Peptoprime
CHMWC

SEQ ID NO: 84 - protein, 5 aa, Artificial Sequence
  5-mer Peptoprime
WQMWC

SEQ ID NO: 85 - protein, 5 aa, Artificial Sequence
  5-mer Peptoprime
WMYWC

SEQ ID NO: 86 - protein, 5 aa, Artificial Sequence
  5-mer Peptoprime
CYYWC

SEQ ID NO: 87 - protein, 5 aa, Artificial Sequence
  5-mer Peptoprime
WHYWC

SEQ ID NO: 88 - protein, 5 aa, Artificial Sequence
  5-mer Peptoprime
WWEWC

SEQ ID NO: 89 - protein, 5 aa, Artificial Sequence
  5-mer Peptoprime
WFHWC

SEQ ID NO: 90 - protein, 5 aa, Artificial Sequence
  5-mer Peptoprime
HMCWC

SEQ ID NO: 91 - protein, 5 aa, Artificial Sequence
  5-mer Peptoprime
WHSWC

SEQ ID NO: 92 - protein, 5 aa, Artificial Sequence
  5-mer Peptoprime
CWQWC

SEQ ID NO: 93 - protein, 5 aa, Artificial Sequence
  5-mer Peptoprime
QFWWC

SEQ ID NO: 94 - protein, 5 aa, Artificial Sequence
  5-mer Peptoprime
MMWWC

SEQ ID NO: 95 - protein, 5 aa, Artificial Sequence
  5-mer Peptoprime
QMWWC

SEQ ID NO: 96 - protein, 5 aa, Artificial Sequence
  5-mer Peptoprime
WHWCI

SEQ ID NO: 97 - protein, 5 aa, Artificial Sequence
  5-mer Peptoprime
MCWWI

SEQ ID NO: 98 - protein, 5 aa, Artificial Sequence
  5-mer Peptoprime
MWHMQ

SEQ ID NO: 99 - protein, 5 aa, Artificial Sequence
  5-mer Peptoprime
WWCHQ

SEQ ID NO: 100 - protein, 5 aa, Artificial Sequence
  5-mer Peptoprime
WCMWQ

SEQ ID NO: 101 - protein, 5 aa, Artificial Sequence
  5-mer Peptoprime
CMWWQ

SEQ ID NO: 102 - protein, 5 aa, Artificial Sequence
  5-mer Peptoprime
WYMMW

SEQ ID NO: 103 - protein, 5 aa, Artificial Sequence
  5-mer Peptoprime
WWMMW

SEQ ID NO: 104 - protein, 5 aa, Artificial Sequence
  5-mer Peptoprime
QMYMW

SEQ ID NO: 105 - protein, 5 aa, Artificial Sequence
  5-mer Peptoprime
WWYMW

SEQ ID NO: 106 - protein, 5 aa, Artificial Sequence
  5-mer Peptoprime
WWTMW

SEQ ID NO: 107 - protein, 5 aa, Artificial Sequence
  5-mer Peptoprime
HMHMW

SEQ ID NO: 108 - protein, 5 aa, Artificial Sequence
  5-mer Peptoprime
CYCMW

SEQ ID NO: 109 - protein, 5 aa, Artificial Sequence
  5-mer Peptoprime
QWCMW

SEQ ID NO: 110 - protein, 5 aa, Artificial Sequence
  5-mer Peptoprime
CEWMW

SEQ ID NO: 111 - protein, 5 aa, Artificial Sequence
  5-mer Peptoprime
HCWMW

SEQ ID NO: 112 - protein, 5 aa, Artificial Sequence
  5-mer Peptoprime
CIWMW

SEQ ID NO: 113 - protein, 5 aa, Artificial Sequence
  5-mer Peptoprime
CQWMW

SEQ ID NO: 114 - protein, 5 aa, Artificial Sequence
  5-mer Peptoprime
WCWYW

SEQ ID NO: 115 - protein, 5 aa, Artificial Sequence
  5-mer Peptoprime
WMCNW

SEQ ID NO: 116 - protein, 5 aa, Artificial Sequence
  5-mer Peptoprime
WYMEW

SEQ ID NO: 117 - protein, 5 aa, Artificial Sequence
  5-mer Peptoprime
CCYEW

SEQ ID NO: 118 - protein, 5 aa, Artificial Sequence
  5-mer Peptoprime
CMWEW

SEQ ID NO: 119 - protein, 5 aa, Artificial Sequence
  5-mer Peptoprime
MQCTW

SEQ ID NO: 120 - protein, 5 aa, Artificial Sequence
  5-mer Peptoprime
WFMHW

SEQ ID NO: 121 - protein, 5 aa, Artificial Sequence
  5-mer Peptoprime
YMCHW

SEQ ID NO: 122 - protein, 5 aa, Artificial Sequence
  5-mer Peptoprime
WIWHW

SEQ ID NO: 123 - protein, 5 aa, Artificial Sequence
  5-mer Peptoprime
WWYKW

SEQ ID NO: 124 - protein, 5 aa, Artificial Sequence
  5-mer Peptoprime
WCWKW

SEQ ID NO: 125 - protein, 5 aa, Artificial Sequence
  5-mer Peptoprime
WYMCW

SEQ ID NO: 126 - protein, 5 aa, Artificial Sequence
  5-mer Peptoprime
HQMCW

SEQ ID NO: 127 - protein, 5 aa, Artificial Sequence
  5-mer Peptoprime
WYPCW

SEQ ID NO: 128 - protein, 5 aa, Artificial Sequence
  5-mer Peptoprime
WWECW

SEQ ID NO: 129 - protein, 5 aa, Artificial Sequence
  5-mer Peptoprime
HMHCW

SEQ ID NO: 130 - protein, 5 aa, Artificial Sequence
  5-mer Peptoprime
WWHCW

SEQ ID NO: 131 - protein, 5 aa, Artificial Sequence
  5-mer Peptoprime
HWKCW

SEQ ID NO: 132 - protein, 5 aa, Artificial Sequence
  5-mer Peptoprime
HTQCW

SEQ ID NO: 133 - protein, 5 aa, Artificial Sequence
  5-mer Peptoprime
MNWCW

SEQ ID NO: 134 - protein, 5 aa, Artificial Sequence
  5-mer Peptoprime
INWCW

SEQ ID NO: 135 - protein, 5 aa, Artificial Sequence
  5-mer Peptoprime
ICWCW

SEQ ID NO: 136 - protein, 5 aa, Artificial Sequence
  5-mer Peptoprime
WCMQW

SEQ ID NO: 137 - protein, 5 aa, Artificial Sequence
  5-mer Peptoprime
CWCDW

SEQ ID NO: 138 - protein, 5 aa, Artificial Sequence
  5-mer Peptoprime
WMFWW

SEQ ID NO: 139 - protein, 5 aa, Artificial Sequence
  5-mer Peptoprime
HHMWW

SEQ ID NO: 140 - protein, 5 aa, Artificial Sequence
  5-mer Peptoprime
CHMWW

SEQ ID NO: 141 - protein, 5 aa, Artificial Sequence
  5-mer Peptoprime
VCYWW

SEQ ID NO: 142 - protein, 5 aa, Artificial Sequence
  5-mer Peptoprime
WCKWW

SEQ ID NO: 143 - protein, 5 aa, Artificial Sequence
  5-mer Peptoprime
WYCWW

SEQ ID NO: 144 - protein, 5 aa, Artificial Sequence
  5-mer Peptoprime
CVCWW

SEQ ID NO: 145 - protein, 5 aa, Artificial Sequence
  5-mer Peptoprime
CKCWW

SEQ ID NO: 146 - protein, 5 aa, Artificial Sequence
  5-mer Peptoprime
CCMWF

SEQ ID NO: 147 - protein, 5 aa, Artificial Sequence
  5-mer Peptoprime
CCNMW

SEQ ID NO: 148 - protein, 5 aa, Artificial Sequence
  5-mer Peptoprime
CDCMW

SEQ ID NO: 149 - protein, 5 aa, Artificial Sequence
  5-mer Peptoprime
CFWMW

SEQ ID NO: 150 - protein, 5 aa, Artificial Sequence
  5-mer Peptoprime
CHWWM

SEQ ID NO: 151 - protein, 5 aa, Artificial Sequence
  5-mer Peptoprime
CKWMC

SEQ ID NO: 152 - protein, 5 aa, Artificial Sequence
  5-mer Peptoprime
CMCCW

SEQ ID NO: 153 - protein, 5 aa, Artificial Sequence
  5-mer Peptoprime
CMMWC

SEQ ID NO: 154 - protein, 5 aa, Artificial Sequence
  5-mer Peptoprime
CMMWH

SEQ ID NO: 155 - protein, 5 aa, Artificial Sequence
  5-mer Peptoprime
CMWQW

SEQ ID NO: 156 - protein, 5 aa, Artificial Sequence
  5-mer Peptoprime
CQHWM

SEQ ID NO: 157 - protein, 5 aa, Artificial Sequence
  5-mer Peptoprime
CWCMF

SEQ ID NO: 158 - protein, 5 aa, Artificial Sequence
  5-mer Peptoprime
CWCMY

SEQ ID NO: 159 - protein, 5 aa, Artificial Sequence
  5-mer Peptoprime
CWCYM

SEQ ID NO: 160 - protein, 5 aa, Artificial Sequence
  5-mer Peptoprime
CWMEM

SEQ ID NO: 161 - protein, 5 aa, Artificial Sequence
  5-mer Peptoprime
CWMYM

SEQ ID NO: 162 - protein, 5 aa, Artificial Sequence
  5-mer Peptoprime
DWCMC

SEQ ID NO: 163 - protein, 5 aa, Artificial Sequence
  5-mer Peptoprime
EMWCM

SEQ ID NO: 164 - protein, 5 aa, Artificial Sequence
  5-mer Peptoprime
HMCWM

SEQ ID NO: 165 - protein, 5 aa, Artificial Sequence
  5-mer Peptoprime
HMMWC

SEQ ID NO: 166 - protein, 5 aa, Artificial Sequence
  5-mer Peptoprime
HMWCM

SEQ ID NO: 167 - protein, 5 aa, Artificial Sequence
  5-mer Peptoprime
HMWMM

SEQ ID NO: 168 - protein, 5 aa, Artificial Sequence
  5-mer Peptoprime
HMWQW

SEQ ID NO: 169 - protein, 5 aa, Artificial Sequence
  5-mer Peptoprime
HNMCW

SEQ ID NO: 170 - protein, 5 aa, Artificial Sequence
  5-mer Peptoprime
IMWCW

SEQ ID NO: 171 - protein, 5 aa, Artificial Sequence
  5-mer Peptoprime
KWCMM

SEQ ID NO: 172 - protein, 5 aa, Artificial Sequence
  5-mer Peptoprime
KWCWT

SEQ ID NO: 173 - protein, 5 aa, Artificial Sequence
  5-mer Peptoprime
KYMCW

SEQ ID NO: 174 - protein, 5 aa, Artificial Sequence
  5-mer Peptoprime
LHMWC

SEQ ID NO: 175 - protein, 5 aa, Artificial Sequence
  5-mer Peptoprime
MWCTM

SEQ ID NO: 176 - protein, 5 aa, Artificial Sequence
  5-mer Peptoprime
MWWMH

SEQ ID NO: 177 - protein, 5 aa, Artificial Sequence
  5-mer Peptoprime
NMCWH

SEQ ID NO: 178 - protein, 5 aa, Artificial Sequence
  5-mer Peptoprime
NMMCW

SEQ ID NO: 179 - protein, 5 aa, Artificial Sequence
  5-mer Peptoprime
NMMWC

SEQ ID NO: 180 - protein, 5 aa, Artificial Sequence
  5-mer Peptoprime
QCWMC

SEQ ID NO: 181 - protein, 5 aa, Artificial Sequence
  5-mer Peptoprime
QMCWW

SEQ ID NO: 182 - protein, 5 aa, Artificial Sequence
  5-mer Peptoprime
WCCWD

SEQ ID NO: 183 - protein, 5 aa, Artificial Sequence
  5-mer Peptoprime
WCIHM

SEQ ID NO: 184 - protein, 5 aa, Artificial Sequence
  5-mer Peptoprime
WCIMM

SEQ ID NO: 185 - protein, 5 aa, Artificial Sequence
  5-mer Peptoprime
WCYCM

SEQ ID NO: 186 - protein, 5 aa, Artificial Sequence
  5-mer Peptoprime
WHIMW

SEQ ID NO: 187 - protein, 5 aa, Artificial Sequence
  5-mer Peptoprime
WHMCM

SEQ ID NO: 188 - protein, 5 aa, Artificial Sequence
  5-mer Peptoprime
WHMKC

SEQ ID NO: 189 - protein, 5 aa, Artificial Sequence
  5-mer Peptoprime
WMCHM

SEQ ID NO: 190 - protein, 5 aa, Artificial Sequence
  5-mer Peptoprime
WNMWW

SEQ ID NO: 191 - protein, 5 aa, Artificial Sequence
  5-mer Peptoprime
WNWHM

SEQ ID NO: 192 - protein, 5 aa, Artificial Sequence
  5-mer Peptoprime
WTWCM

SEQ ID NO: 193 - protein, 5 aa, Artificial Sequence
  5-mer Peptoprime
WWHMC

SEQ ID NO: 194 - protein, 5 aa, Artificial Sequence
  5-mer Peptoprime
WWMHM

SEQ ID NO: 195 - protein, 5 aa, Artificial Sequence
  5-mer Peptoprime
YCCWM

SEQ ID NO: 196 - protein, 5 aa, Artificial Sequence
  5-mer Peptoprime
CMWNC

SEQ ID NO: 197 - protein, 5 aa, Artificial Sequence
  5-mer Peptoprime
CWMEW

SEQ ID NO: 198 - protein, 5 aa, Artificial Sequence
  5-mer Peptoprime
MCWYH

SEQ ID NO: 199 - protein, 10 aa, Artificial Sequence
  Peptide 9R
RRRRRNWMWC

SEQ ID NO: 200 - protein, 5 aa, Artificial Sequence
  9S1
WCMNW

SEQ ID NO: 201 - protein, 10 aa, Artificial Sequence
  Peptide 9S1R
RRRRRWCMNW

(19) United States
(12) Patent Application Publication
(10) Pub. No.: US 2014/0249293 A1
(43) Pub. Date: Sep. 4, 2014

(54) Absent and Rare Peptides and Therapeutic Uses Thereof

(71) Applicant: Greg Hampikian, Boise, ID (US)

(72) Inventor: Greg Hampikian, Boise, ID (US)

(73) Assignee: Boise State University, Bosie, ID (US)

(21) Appl. No.: 13/358,952

(22) Filed: Jan. 26, 2012

(51) Int. Cl. G06F 17/30; C07K 7/06

(52) U.S. Cl. CPC G06F 17/30477; C07K 7/06
USPC 530/322; 435/29; 435/5; 530/330; 530/391.1; 530/350; 707/769

(57) ABSTRACT

The present invention relates to methods for searching and identifying absent and rare peptide sequences from public databases and their uses in the treatment of pathological diseases. One embodiment of the present invention provides a method that includes: a) storing, in a memory or storage of a computing device, a set of at least one peptide sequence of fixed length; b) searching, by the computing device, for a peptide sequence from the set within at least one database having naturally-occurring amino acid sequences; and c) classifying, by the number of appearance in the database, the peptide sequence.

### FEDERAL FUNDING LEGEND

This invention was made with government support under DOD Grant Number W81XWH-10-1-0097. The Government has certain rights to this invention.

### BACKGROUND

The present invention relates to bioinformatics and peptide therapeutic agents. In particular, the present invention relates to methods for searching and identifying absent and rare peptide sequences from public databases and their uses in the treatment of pathological diseases.

One of the primary goals of drug discovery is to identify biologically active compositions that have practical clinical utility. A great deal of effort is typically used to discover bioactive small molecules, and among these, small peptides can be very effective therapeutic agents. Compared to other small molecule agents, peptides typically show higher specificity, greater potency, and fewer toxicology problems. Peptides also tend not to accumulate in organs or face drug-to-drug interaction challenges.

Because peptides are active regulators that are involved in most physiological processes, they can potentially be used as drugs or as lead structures to develop peptidomimetics. For example, short peptide aptamers (˜10-20 amino acids), which are typically variable peptide loops, have been inserted into scaffold proteins to be used as inhibitors with high affinity. The loops are generally designed to interfere with other protein interactions inside the cell. In some cases, these interactions may be critical to a disease-associated protein function. Peptide aptamers typically do not occur naturally but may be identified using methods such as, but not limited to, high-throughput screening methods. In some cases, the binding affinity of aptamers are comparable to antibodies.

As used herein, the term “short peptide” generally refers to peptides having a sequence of about 50 amino acids or less. This term may be somewhat context dependent. In some cases, the upper limit length of a short peptide may depend on practical considerations such as, but not limited to, peptide synthesis technology, computational technology (i.e., computing power required to generate a library of peptide sequences), and the like. Generally, a “short peptide” may be considered non-natural if its isolated form does not occur naturally. Thus, in some cases, the sequence of a non-natural short peptide may occur naturally as part of a longer naturally-occurring peptide or protein.

In some cases, small degradation products of proteins can have bioactive regulatory effects. Recent studies have investigated the role of peptides derived from the pregnancy hormone human chorionic gonadotropin (hCG). The short peptides were designed, in large part, according to the known nick sites in loop-2 of β-hCG. These short peptides (˜3-7 amino acids) have demonstrated some ability to inhibit severe inflammation, onset of type 1 diabetes, renal failure and tumorigenesis. In particular, 4-mer (AQGV) has shown promise in accelerating the recuperation of mice after lethal exposure to radiation.

In some cases, short peptides are also known to elicit or facilitate immunological responses inside a human body. For example, when a foreign protein invades a host cell, they are processed and presented on a major histocompatibility complex (MHC) molecule, which is found on every nucleated cell of the body. The function of MHC molecules is to display fragments of foreign proteins (epitopes) from within the cell to T cells. Healthy cells are ignored but cells containing foreign peptides are attacked by the immune system. T cell epitopes presented by MHC class I molecules are typically peptides between 8 and 11 amino acids in length. MHC class II molecules typically present longer peptides. Thus, certain disease-specific epitopes have the potential to be utilized as therapeutic agents that elicit or suppress immunogenic responses.

Potential peptide drug candidates are often screened using high-throughput assays that test for bioactivity. These high-throughput assays generally involve the synthesis of a large library of peptides that are introduced into an array. Each spot on the array has a unique peptide that is assayed for biological activity (e.g., ability to bind to a target molecule).

While these assays have been successful in identifying bioactive peptides such as antimicrobial peptides, receptor agonists and antagonists, protein kinase inhibitors, T-cell epitopes, there are several disadvantages to a high-throughput approach. For instance, high-throughput approaches typically require the synthesis of a large number of peptides. In other words, the combination of the naturally occurring amino acids necessitates the construction of a very large library of different peptide sequences even for short peptides. For example, there are 3,200,000 unique peptide sequences that can arise from a peptide containing only 5 amino acids. The number of unique peptide sequences grows 20-fold with each additional amino acid. The synthesis and high-throughput assaying of large libraries of peptides, while faster than traditional drug discovery approaches, may still be a time consuming and costly process.

### SUMMARY OF THE INVENTION

The present invention relates to bioinformatics and peptide therapeutic agents. In particular, the present invention relates to methods for searching and identifying absent and rare peptide sequences from public databases and their uses in the treatment of pathological diseases.

In some embodiments, the present invention provides methods comprising: a) storing, in a memory or storage of a computing device, a set of at least one peptide sequence of fixed length; b) searching, by the computing device, for a peptide sequence from the set within at least one database comprising naturally-occurring amino acid sequences; and c) classifying, by the number of appearances in the database, the peptide sequence.

In other embodiments, the present invention provides methods comprising: a) generating, by a computing device, a set of at least one peptide sequence of fixed length; b) searching, by the computing device, for a peptide sequence from the set within at least one database comprising naturally-occurring amino acid sequences; c) classifying, by the number of appearances in the database, the peptide sequence as being one of: a rare peptide or an absent peptide; and d) contacting the rare peptide, the absent peptide, or a scrambled sequence thereof with an organism, a cell, or a virus.

In still other embodiments, the present invention provides synthetic or isolated peptides having a sequence selected from the group consisting of SEQ ID NOS: 1-201.

The features and advantages of the present invention will be readily apparent to those skilled in the art upon a reading of the description of the preferred embodiments that follows.

### BRIEF DESCRIPTION OF THE DRAWINGS

The following figures are included to illustrate certain aspects of the present invention, and should not be viewed as exclusive embodiments. The subject matter disclosed is capable of considerable modification, alteration, and equivalents in form and function, as will occur to those skilled in the art and having the benefit of this disclosure.

FIGS. 1A-1C show photographs of HUT 102 cells treated with a peptide in accordance with the embodiments of this disclosure.

FIG. 2 shows a plot showing the results of an MTT assay of LnCap cells treated with peptide in accordance with the embodiments of this disclosure.

FIG. 3 shows a plot showing the results of an MTT assay of MDA-MB-231 cells treated with peptide in accordance with the embodiments of this disclosure.

FIG. 4 shows a plot showing the results of an MTT assay of W138 cells treated with peptide in accordance with the embodiments of this disclosure.

FIG. 5 shows a photograph of healthy LnCap cells during an MTT assay.

FIG. 6 shows a photograph of LnCap cells treated with a peptide in accordance with the embodiments of this disclosure.

FIG. 7 shows a photograph of LnCap cells treated with a peptide in accordance with the embodiments of this disclosure.

FIG. 8 shows a photograph of LnCap cells treated with a peptide in accordance with the embodiments of this disclosure.

FIGS. 9A-9C show photographs of LnCap cells stained with dyes (FIGS. 9A-9B). FIG. 9C shows a merged image of FIGS. 9A-9B.

FIGS. 10A-10C show photographs of LnCap cells treated with a peptide in accordance with the embodiments of this disclosure and stained with dyes (FIGS. 10A-10B). FIG. 10C shows a photograph of a merged image of FIGS. 10A and 10B.

FIG. 11A-11C show photographs of HUT-102 cells stained with dyes (FIGS. 11A-11B). FIG. 11C shows a merged image of FIGS. 11A and 11B.

FIG. 12A-12C show photographs of HUT-102 cells treated with a peptide in accordance with the embodiments of this disclosure and stained with dyes (FIGS. 12A-12C). FIG. 12C shows a merged image of FIGS. 12A and 12B.

FIG. 13 shows a plot of relative fluorescence intensity in a mitochondrial health assay of LnCap cells after incubation with peptides in accordance with the embodiments of this disclosure.

FIG. 14 shows a plot of relative fluorescence intensity in a mitochondrial healthy assay of LnCap cells after incubation with peptides in accordance with the embodiments of this disclosure.

FIG. 15 shows a plot of relative fluorescence intensity in a mitochondrial healthy assay of LnCap cells after incubation with peptides in accordance with the embodiments of this disclosure.

FIG. 16 shows a plot of relative fluorescence intensity in a mitrochondrial health assay of paclitaxel-resistant LnCap cells treated with paclitaxel and a peptide in accordance with the embodiments of this disclosure.

### DETAILED DESCRIPTION

The present invention relates to bioinformatics and peptide therapeutic agents. In particular, the present invention relates to methods for searching and identifying absent and rare peptide sequences from public databases and their uses in the treatment of pathological diseases.

As used herein, an “absent peptide sequence” refers to a string of amino acids (i.e., a peptide sequence) that is not found in at least one species during a sequence search of the string in publically available genomic databases comprising naturally-occurring peptide and protein sequences. For example, the publically available databases can be the large National Center for Biotechnology Information (NCBI) database or its equivalents. In some cases, a peptide sequence may be absent from publically available genomic databases, which include putative protein translations of DNA sequences. The shortest length sequence or sequences that are absent from a species or species group in the publically available databases will herein be referred to as “nullopeps.” In some cases, a sequence may be absent from only one species or several species. The shortest length sequence or sequences that are absent in all species as known in existing databases will herein be referred to as “peptoprimes.”

Sequences that are present in less than about 5 species will herein be referred to as “rare peptides.” In some cases, a peptide sequence that is initially classified as a peptoprime may later be identified as a rare peptide. In such cases, the rare peptide is referred herein as a “past peptoprime.”

Sequences that are found in about 5 or more species will herein be referred to as “common peptides.”

There are a number of advantages related to the present invention, only a few of which will be discussed herein. The present invention provides short peptide sequences (e.g., absent and rare peptide sequences) that are potentially bioactive. Without wishing to be limited by theory, it is believed that at least some of the absent and rare peptide sequences or derivative sequences thereof may be bioactive (e.g., cytotoxic, immunogenic, growth or metabolism inhibitors) as these sequences may have been evolutionarily excluded from certain species. Consequently, these small peptide sequences may be useful as therapeutic agents. As therapeutic agents, peptides typically show higher specificity, greater potency, and fewer toxicology problems compared to other small molecules.

The present invention provides methods for searching and identifying absent and rare peptide sequences from protein sequence databases. A protein sequence database is any large collection of protein sequence data stored on a computer. In some cases, the protein sequences may be stored locally on a computer or they may be stored remotely on, for example, a server. The protein sequence database may include sequences from a single organism, or it can include sequences from multiple organisms. Suitable examples of protein sequence databases include, but are not limited to, the GenBank Protein database maintained by the National Center for Biotechnology Information (on the world wide web at ncbi.nlm.nih.gov), the Universal Protein Resource (UniProt), maintained by the European Bioinformatics Institute (EBI), the Swiss Institute of Bioinformatics (SIB) and the Protein Information Resource (PIR) (on the world wide web at uniprot.org), and the ExPASy bioinformatics resource portal, made available through the SIB (on the world wide web at expasy.org/proteomics).

The protein sequence databases comprise naturally-occurring peptide and/or protein sequences. In some cases, the databases may contain “hypothetical” proteins whose existence have yet to be verified. Once identified according to embodiments described herein, absent and rare peptide sequences may be assayed for their bioactivity to identify specific sequences that may be particularly useful in numerous applications such as, but not limited to, therapeutic agents in the treatment of pathological diseases, insecticides, herbicides, biocides and other killing agents, and agents that induce stasis such a bacteriostatins. In some cases, the absent and rare peptide sequences may be used as agents that can reverse drug resistance, adjuvants to enhance vaccines, tags to mark engineered or recombinant proteins, enhancers of other drugs, drug delivery agents, carriers, transfection and transmembrane delivery agents, immune suppressors, appetite suppressants, biological glues, artificial threads, bandages, binders, adhesives, gels and matrices for cell support, coatings for tissue culture plastice, and the like.

Most peptide drug discovery strategies involve the use of high-throughput screening methods in which a library of thousands if not hundreds of thousands of peptides are generated. The present invention can bypass conventional high-throughput peptide drug discovery strategies by identifying absent and rare peptide sequences that may have evolutionarily excluded from certain species because of their bioactivity. This may save considerable time as well as cost.

The methods of the present invention may be generally practiced using a computer system. In certain aspects, the computer system may be implemented using hardware or a combination of software and hardware, either in a dedicated server, or integrated into another entity, or distributed across multiple entities.

Computer system (or “computing device”) can include, in addition to hardware, code that creates an execution environment for the computer program in question, e.g., a code that constitutes processor firmware, a protocol stack, a database management system, an operating system, or a combination of one or more of them stored in an included memory, such as a Random Access Memory (RAM), a flash memory, a Read Only Memory (ROM), a Programmable Read-Only Memory (PROM), an Erasable PROM (EPROM), register, a hard disk, a removable disk, a CD-ROM, a DVD, or any other suitable storage device, coupled to a bus for storing information and instructions to be executed by a processor. The processor and the memory can be supplemented by, or incorporated in, special purpose logic circuitry.

The instructions may be stored in the memory and implemented in one or more computer program products, i.e., one or more modules of computer program instructions encoded on a computer readable medium for execution by, or to control the operation of, the computer system, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory may also be used for storing temporary variable or other intermediate information during execution of instructions to be executed by a processor.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

The present invention provides peptides comprising a sequence from one of SEQ ID NO: 1-201. While these peptides are preferably prepared using synthetic methods (e.g., peptide synthesizers), if possible, these peptides may also be isolated from organisms. In some embodiments, the synthetic or isolated peptide comprises a sequence that is scrambled from SEQ ID NO: 1-201.

As used herein, a “scrambled sequence” or a “sequence scrambled from a ‘parent’ sequence” generally refers to a sequence that contains the same amino acids as the parent sequence but the order of amino acids has been altered. For example, “ALKMV” and “LKVMA” are scrambled sequences of “KLAVM.”

In some embodiments, the synthetic or isolated peptide may further comprise a modification that enhances, impairs, or blocks cell surface binding of the peptide; imparts detection capability, improves bioavailabity; improves solubility; enhances cellular uptake of the peptide; enhances, impairs, or blocks the binding of the peptide to a cell surface protein; enhances, impairs, or blocks activation of an immunological response; enhances, impairs, or blocks mitochondrial impairment; and the like. Such modification can take the form of a composition selected from the group consisting of: a second peptide, a pharmaceutical compound, a growth factor, a primer, a nucleic acid, a carbohydrate, a metal, an antigen, an adjuvant, an antibody, a fluorescent molecule, a carbohydrate, a nucleotide, a lipid, an arginine or a string of arginines, a synthetic or natural polymer, a biomarker, and combinations thereof. In some embodiments, the composition may be bonded (e.g., covalently) to the synthetic or isolated peptide. In some embodiments, the composition may be coordinated or complexed to the synthetic or isolated peptide.

Exemplary modifications include, but are not limited to, an addition of 2 or more arginines to the N-terminal or C-terminal end of the peptide.

Exemplary immunological responses include, but are not limited to, activation or suppression of: a B cell, a B Cell progenitor, a T cell, a T cell progenitor, a mast cell, a langerhans cell, a dendritic cell, a natural killer cell, a granulocyte-monocyte progenitor, a monocyte, a neutrophil, an eosinophil, a basophil, a magakaryocyte, a platelet, an erythrocyte, an erythroid progenitor cell and combinations thereof.

The present invention provides methods comprising: a) storing, in a memory or storage of a computing device, a set of peptide sequences of fixed length; b) searching for the peptide sequences from the set within at least one database comprising naturally-occurring amino acid sequences; and c) classing, by the number of appearances in the database, the peptide sequence. In some embodiments, the peptide sequence is classified as a naturally-occurring peptide, a rare peptide, a nullopep, or a peptoprime. Optionally, the method may further comprise: d) regenerating, by the computing device, the set of peptide sequences wherein the fixed length is increased; and e) repeating b), c), and d). Optionally, the method may further comprise: repeating a), b), and c) after the database has been updated. Optionally, the method may further comprise: assaying the rare peptide, the nullopep, the peptoprime, or scrambled sequences thereof for biological activity.

In some embodiments, the set comprises all possible peptide sequences of the fixed length. The peptide sequence may generally be of any fixed length, for example, but not limited to, between about 2 to about 50 amino acids. In one preferred embodiment, the fixed length is between about 2 to about 20 amino acids. In some cases, the upper limit may be a function of available computing power and storage capacity. In some embodiments, the sequences may be culled by calculating the expected frequency of the sequence given the frequency of the individual amino acids, and the sub-sequences that are contained within the absent sequence. In some embodiments, those sequences which have a higher theoretical probability based on the frequency of their sub sequences may be selected for storage and/or further study.

In some embodiments, b) and c) are repeated until all peptide sequences within the set have been searched in the database.

The present invention provides methods comprising: a) generating, by a computing device, a set of peptide sequences of fixed length; b) searching for the peptide sequences from the set within at least one database, stored on a server, comprising naturally-occurring amino acid sequences; c) identifying, by the computing device, each peptide sequence as a naturally-occurring peptide, a rare peptide, a nullopep, or a peptoprime; and d) contacting the rare peptide, the nullopep, the peptoprime, or scrambled sequences thereof with an organism, a cell or a virus.

In some embodiments, the generated sequences may be prioritized for searching. For example, certain sequences may be prioritized based on factors such as, but not limited to, hydrophobicity/hydrophilicity, PI, molecular weight, predicted structure (alpha helix, beta sheet, barrel, etc.), predicted immunogenicity, and the percentage of particular amino acids such as arginine, methionine, and cysteine.

In some embodiments, the peptide is an epitope. In related embodiments, the peptide is a conformation or linear epitope that interacts directly with an immune cell. In some embodiments, the peptide may be modified to enhance cell surface binding of the peptide or a composition selected from the group: an antibody, an antigen, a fluorescent molecule, a carbohydrate, a nucleic acid, a lipid, a metal, a glass, a silicate, a mineral, a radioisotope, and any combination thereof. In some embodiments, the peptide may be modified to enhance cellular uptake of itself or a composition selected from the group consisting of: a pharmaceutical compound, a growth factor, a primer, a biomarker, a nucleic acid, a lipid, a carbohydrate, a metal, a glass, a silicate, a mineral, a radioisotope, a dye, an antigen, an adjuvant, and combinations thereof.

In some embodiments, the cell has a resistance to a drug, chemotherapeutic agent, phage therapy or pesticide composition such as, but not limited to, taxol, methicillin, vancomycin, fluorouracil, irinotecan, oxaliplatin, cisplatin, carboplatin, fluoroquinolone, isoniazid, rifampin, insulin, chloroquine, bacilius thuringiensis, multidrug, and the like. In some embodiments, the cell has a pathological disease. In some embodiments, the pathological disease comprises a cancer selected from the group consisting of: breast cancer, prostate cancer, leukemia, melanoma, and combinations thereof.

To facilitate a better understanding of the present invention, the following examples of preferred embodiments are given. In no way should the following examples be read to limit, or to define, the scope of the invention.

### Example 1

In one example, 198 5-amino acid sequences were initially identified according to some embodiments of the present invention. The sequences are shown in Table 1 below. These sequences were initially identified as peptoprimes. Some of these sequences have since been reported in databases. However, these reported sequences (“past peptoprimes”) may still be considered rare and, in some cases, may still be absent (some of these sequences have been identified in “hypothetical proteins” and are not yet verified).

Several of the 198 initially identified peptoprimes were extremely difficult to solubilize. In some cases, this was overcome by using a carbohydrate and adding 5 arginine residues to the 5-amino acid peptoprimes (resulting in a total sequence length of 10 amino acids). It is also believed that the arginines enhance the peptide's ability to penetrate cells.

TABLE 1
5-amino acid sequences.

| Sequence | SEQ ID NO. | Comments |
|---|---|---|
| WMWCW | 1 | |
| MWCWM | 2 | |
| MWCWY | 3 | |
| WMWCN | 4 | |
| WMWCE | 5 | |
| MWCWT | 6 | |
| MWMWC | 7 | |
| YWMWC | 8 | |
| NWMWC | 9 | Peptide 9 |
| MWCWQ | 10 | |
| WMCYH | 11 | |
| WMCQY | 12 | |
| MWMCT | 13 | |
| HFWMC | 14 | |
| WYWMC | 15 | |
| QNWMC | 16 | |
| MQWMC | 17 | |
| DQWMC | 18 | |
| WMCHI | 19 | |
| WMCSW | 20 | |
| CECWF | 21 | |
| WDCWF | 22 | |
| MWCHM | 23 | |
| WCQHM | 24 | |
| DCWHM | 25 | |
| WCPCM | 26 | |
| WWYCM | 27 | |
| WHHCM | 28 | |
| WMCCM | 29 | |
| QWCCM | 30 | |
| WWQCM | 31 | |
| MFWCM | 32 | |
| NWWCM | 33 | |
| MCHWM | 34 | |
| CMCWM | 35 | |
| HWCWM | 36 | |
| HMWWM | 37 | |
| MWHWP | 38 | |
| NWCWP | 39 | |
| WWIMY | 40 | |
| MWCHY | 41 | |
| CWMWY | 42 | |
| WCTWY | 43 | |
| MCHWY | 44 | |
| WHCWY | 45 | |
| CNWWY | 46 | |
| MCWWY | 47 | |
| MCMCN | 48 | |
| WCMWE | 49 | |
| WCMWV | 50 | |
| HCWHT | 51 | |
| MWHCT | 52 | |
| MWWCT | 53 | |
| WWCQT | 54 | |
| NWCMH | 55 | |
| CMWPH | 56 | |
| CMWHH | 57 | |
| WYMCH | 58 | |
| MCWDH | 59 | |
| HHMWH | 60 | |
| CWMWH | 61 | |
| TCWWH | 62 | |
| WCWWH | 63 | |
| WCMMC | 64 | |
| MWYMC | 65 | |
| MWHMC | 66 | |
| EWCMC | 67 | |
| WWCMC | 68 | |
| WHWMC | 69 | |
| WYNPC | 70 | |
| WWHPC | 71 | |
| MMWYC | 72 | |
| CWMHC | 73 | |
| WEYHC | 74 | |
| WWCKC | 75 | |
| CWWKC | 76 | |
| WMYCC | 77 | |
| MWYCC | 78 | |
| HDWCC | 79 | |
| CWWQC | 80 | |
| HYMWC | 81 | |
| YHMWC | 82 | |
| CHMWC | 83 | |
| WQMWC | 84 | |
| WMYWC | 85 | |
| CYYWC | 86 | |
| WHYWC | 87 | |
| WWEWC | 88 | |
| WFHWC | 89 | |
| HMCWC | 90 | |
| WHSWC | 91 | |
| CWQWC | 92 | |
| QFWWC | 93 | |
| MMWWC | 94 | |
| QMWWC | 95 | |
| WHWCI | 96 | |
| MCWWI | 97 | |
| MWHMQ | 98 | |
| WWCHQ | 99 | |
| WCMWQ | 100 | |
| CMWWQ | 101 | |
| WYMMW | 102 | |
| WWMMW | 103 | |
| QMYMW | 104 | |
| WWYMW | 105 | |
| WWTMW | 106 | |
| HMHMW | 107 | |
| CYCMW | 108 | |
| QWCMW | 109 | |
| CEWMW | 110 | |
| HCWMW | 111 | |
| CIWMW | 112 | |
| CQWMW | 113 | |
| WCWYW | 114 | |
| WMCNW | 115 | |
| WYMEW | 116 | |
| CCYEW | 117 | |
| CMWEW | 118 | |
| MQCTW | 119 | |
| WFMHW | 120 | Peptide 120 |
| YMCHW | 121 | |
| WIWHW | 122 | |
| WWYKW | 123 | |
| WCWKW | 124 | |
| WYMCW | 125 | |
| HQMCW | 126 | |
| WYPCW | 127 | |
| WWECW | 128 | |
| HMHCW | 129 | |
| WWHCW | 130 | |
| HWKCW | 131 | |
| HTQCW | 132 | |
| MNWCW | 133 | |
| INWCW | 134 | |
| ICWCW | 135 | |
| WCMQW | 136 | |
| CWCDW | 137 | |
| WMFWW | 138 | |
| HHMWW | 139 | |
| CHMWW | 140 | |
| VCYWW | 141 | |
| WCKWW | 142 | |
| WYCWW | 143 | |
| CVCWW | 144 | |
| CKCWW | 145 | |
| CCMWF | 146 | |
| CCNMW | 147 | |
| CDCMW | 148 | |
| CFWMW | 149 | |
| CHWWM | 150 | |
| CKWMC | 151 | |
| CMCCW | 152 | |
| CMMWC | 153 | |
| CMMWH | 154 | |
| CMWQW | 155 | |
| CQHWM | 156 | |
| CWCMF | 157 | |
| CWCMY | 158 | |
| CWCYM | 159 | |
| CWMEM | 160 | |
| CWMYM | 161 | |
| DWCMC | 162 | |
| EMWCM | 163 | |
| HMCWM | 164 | |
| HMMWC | 165 | |
| HMWCM | 166 | |
| HMWMM | 167 | |
| HMWQW | 168 | |
| HNMCW | 169 | |
| IMWCW | 170 | |
| KWCMM | 171 | |
| KWCWT | 172 | |
| KYMCW | 173 | |
| LHMWC | 174 | |
| MWCTM | 175 | |
| MWWMH | 176 | |
| NMCWH | 177 | |
| NMMCW | 178 | |
| NMMWC | 179 | |
| QCWMC | 180 | |
| QMCWW | 181 | |
| WCCWD | 182 | |
| WCIHM | 183 | |
| WCIMM | 184 | |
| WCYCM | 185 | |
| WHIMW | 186 | |
| WHMCM | 187 | |
| WHMKC | 188 | |
| WMCHM | 189 | |
| WNMWW | 190 | |
| WNWHM | 191 | |
| WTWCM | 192 | |
| WWHMC | 193 | |
| WWMHM | 194 | |
| YCCWM | 195 | |
| CMWNC | 196 | |
| CWMEW | 197 | |
| MCWYH | 198 | |
| RRRRRNWMWC | 199 | Peptide 9R |
| WCMNW | 200 | Peptide 9S1 |
| RRRRRWCMNW | 201 | Peptide 9S1R |

### Example 2

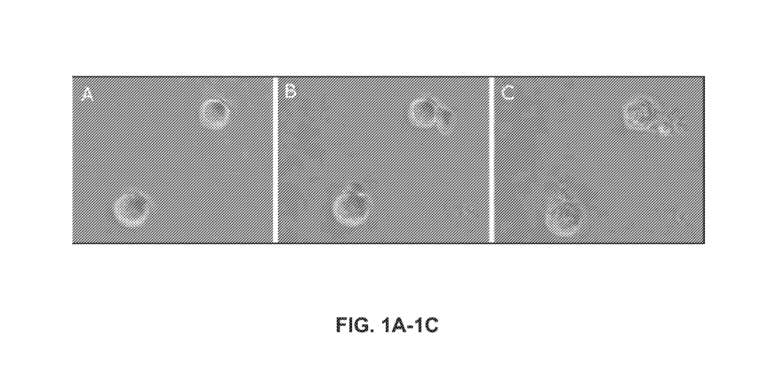

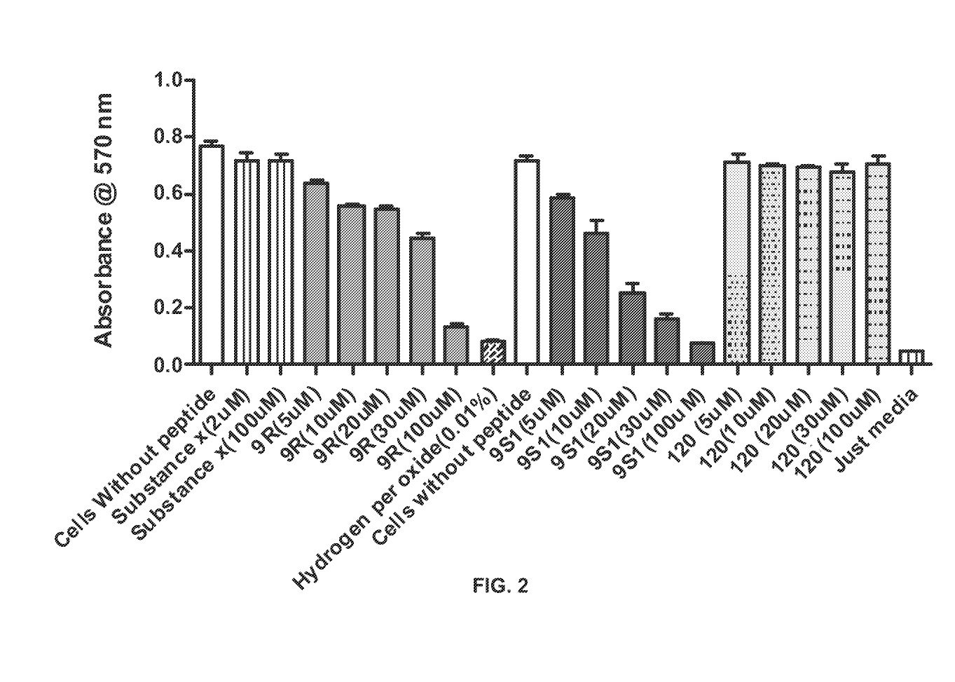

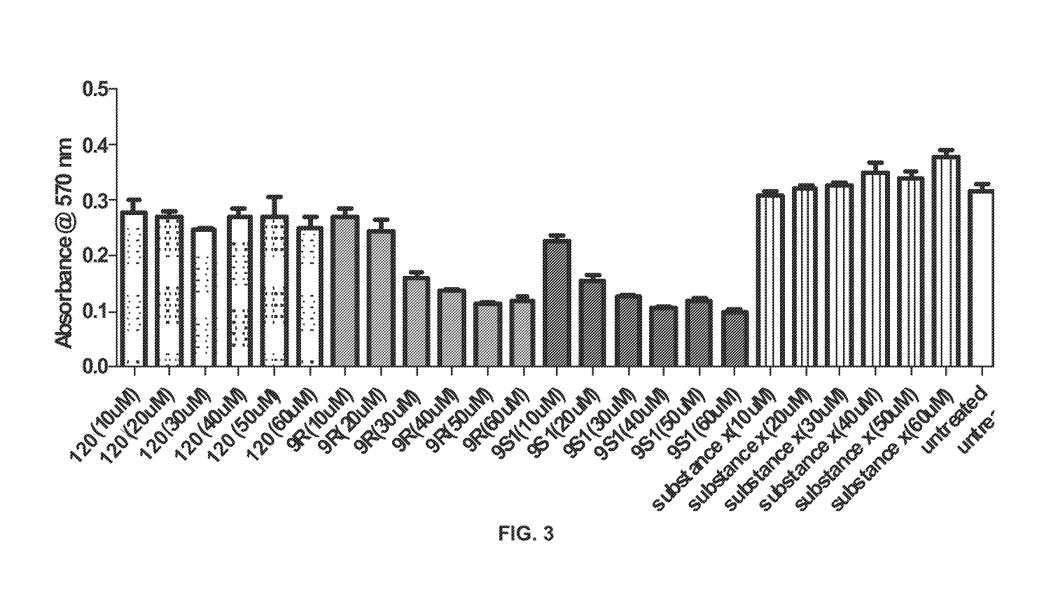

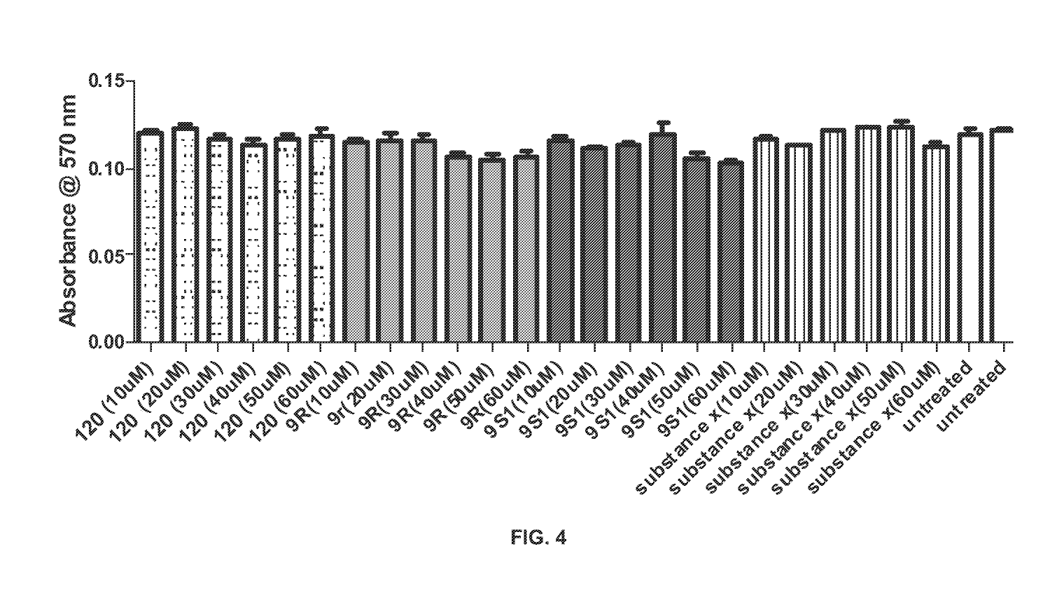

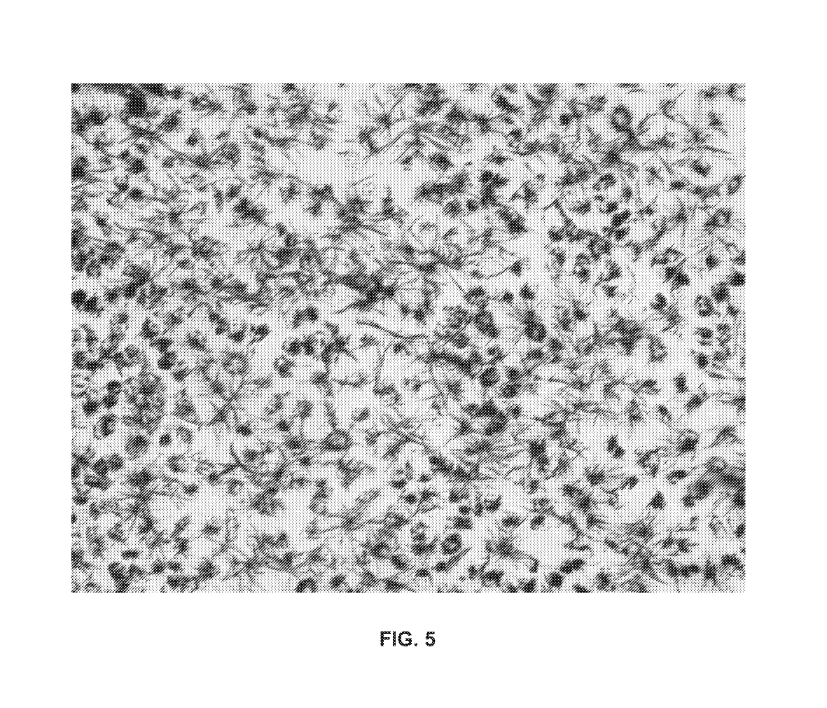

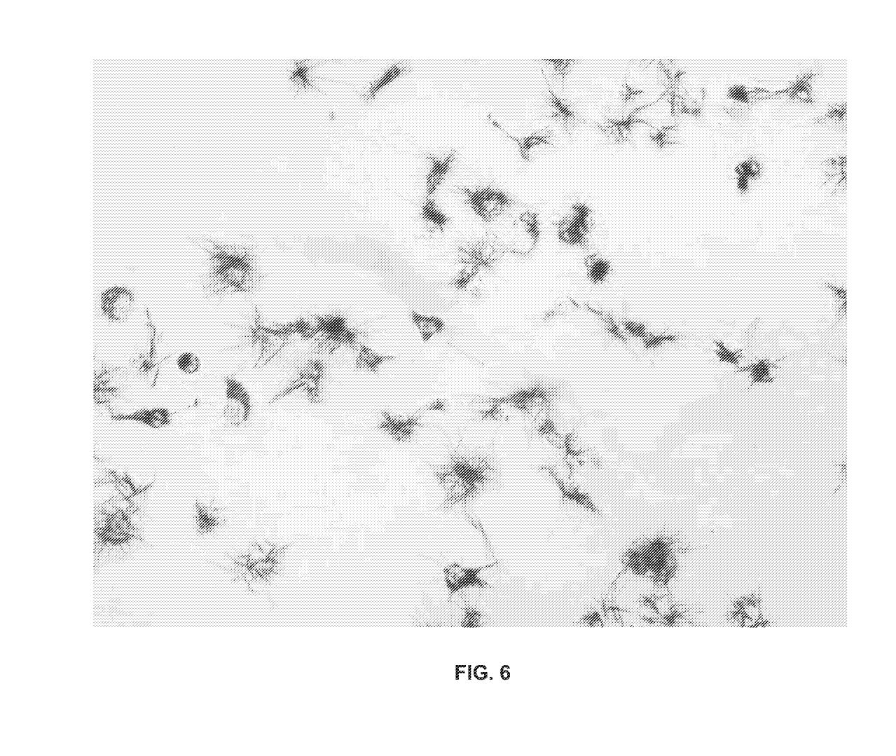

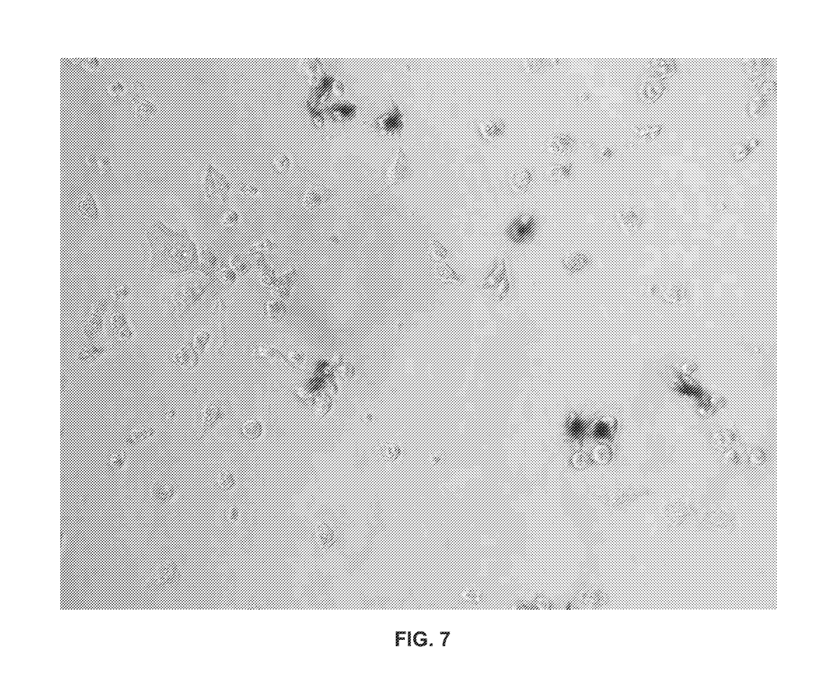

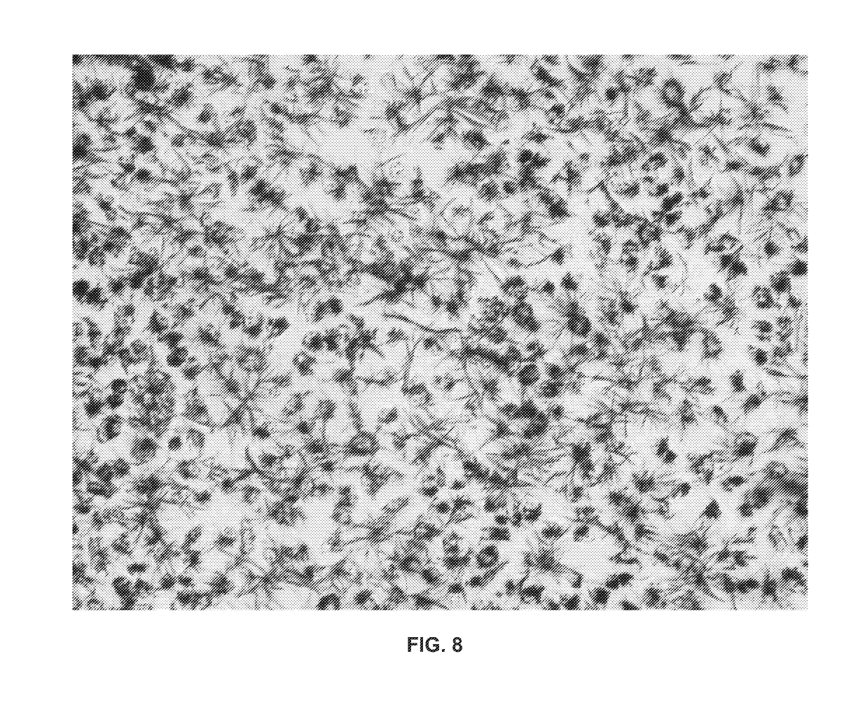

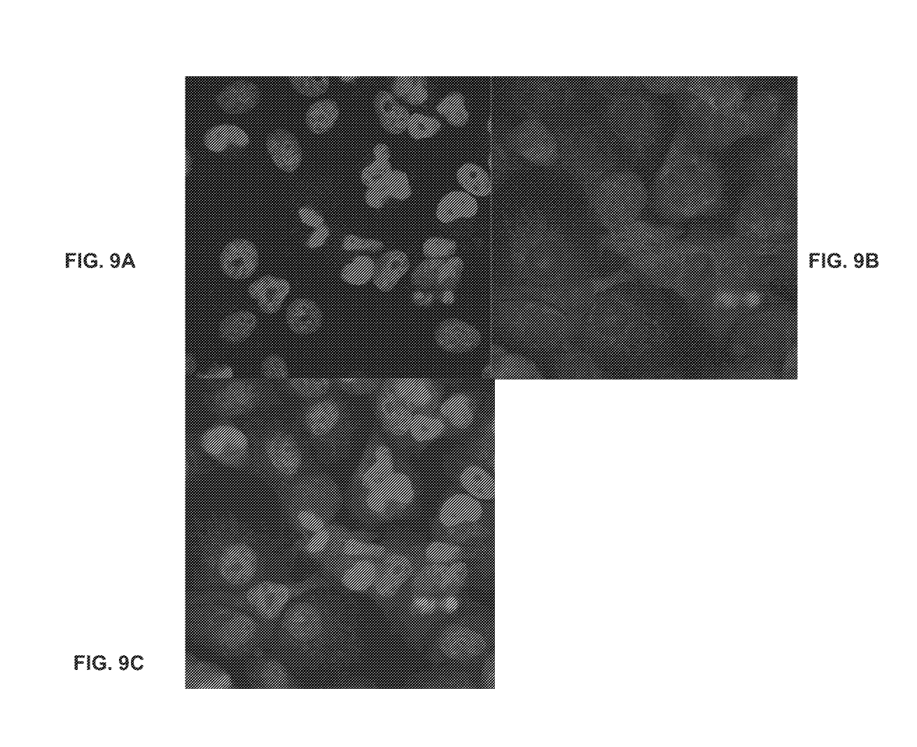

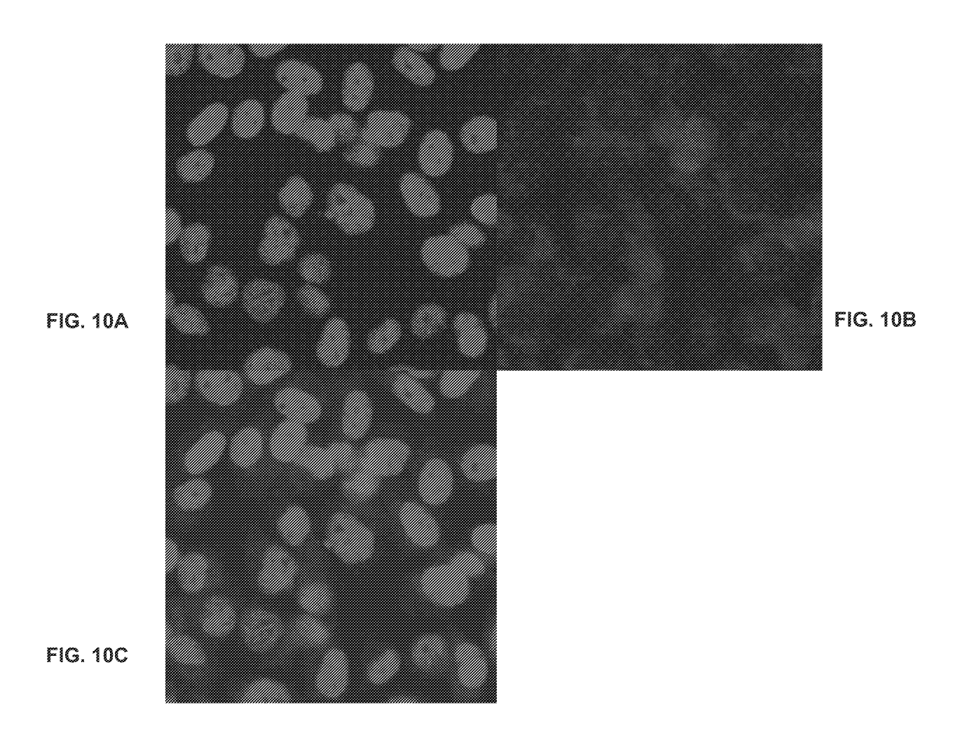

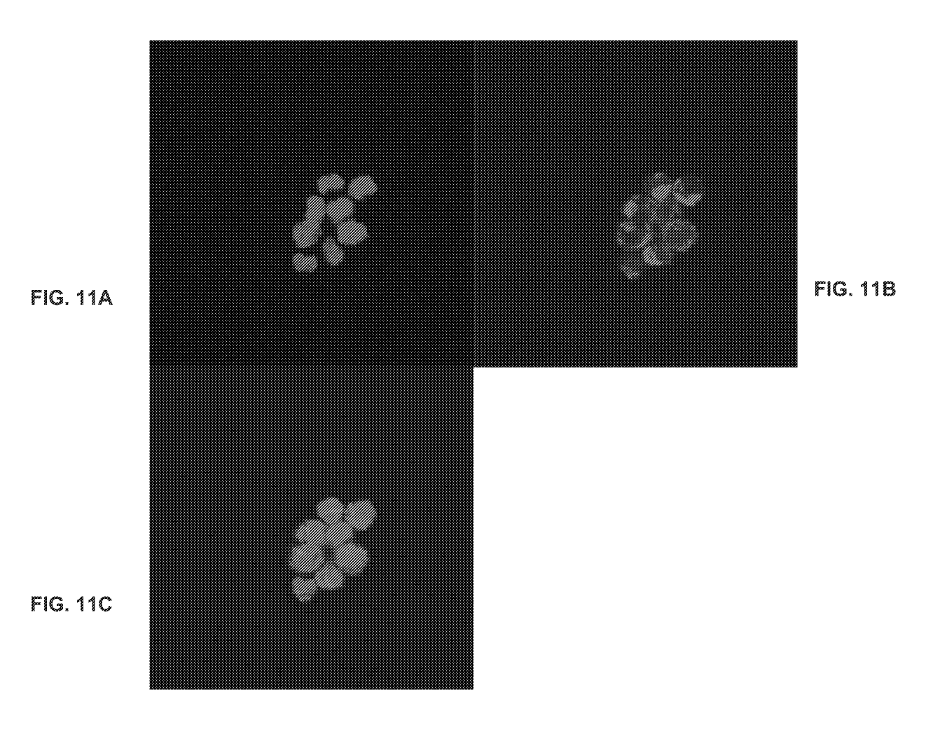

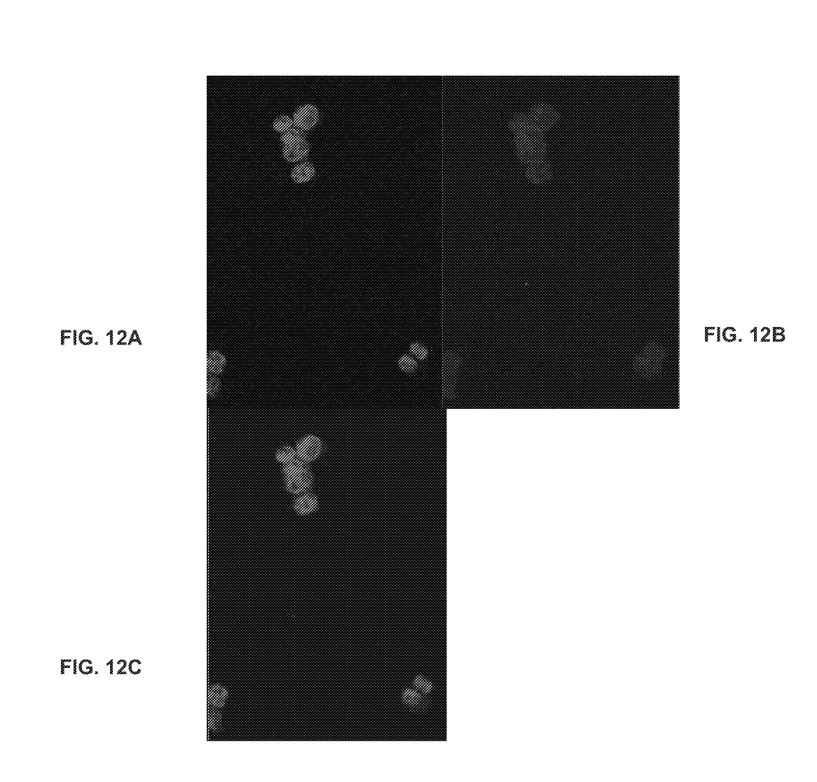

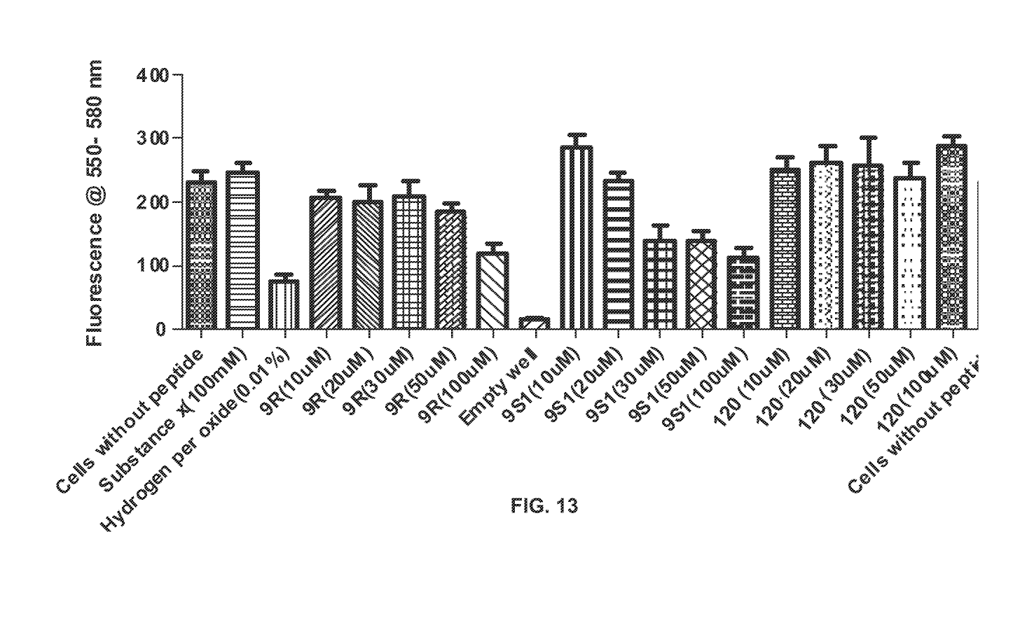

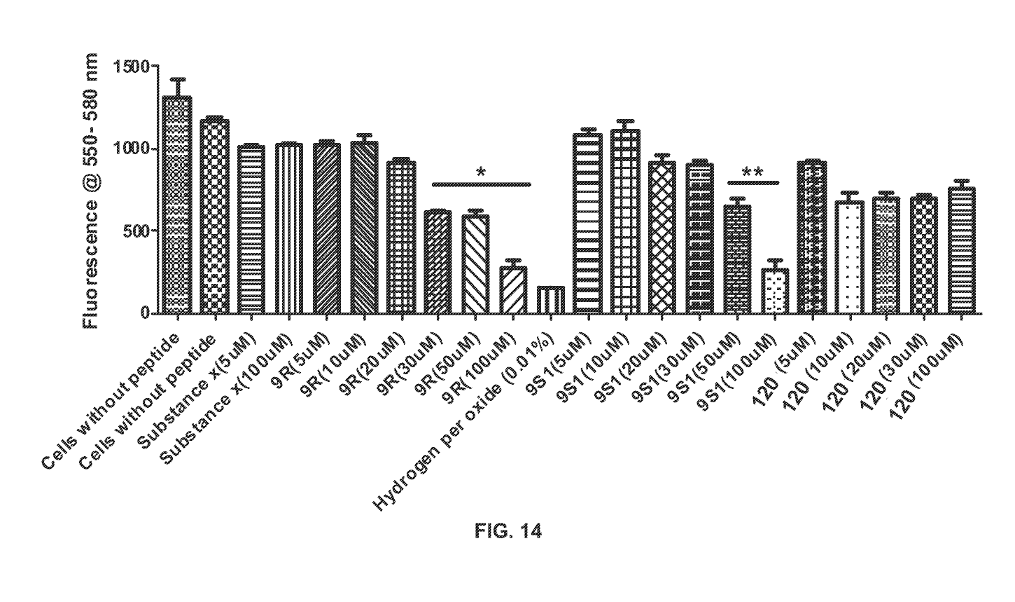

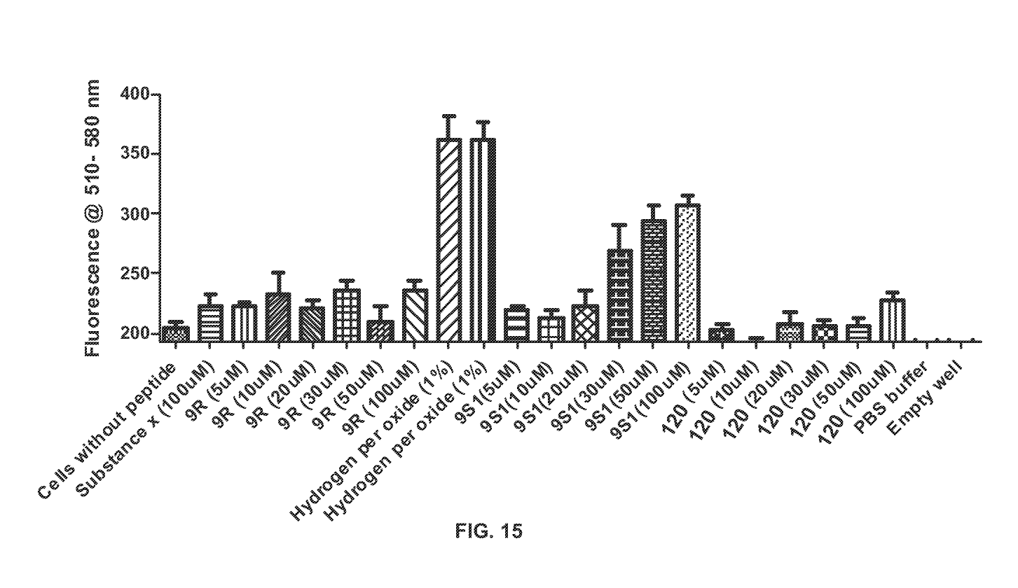

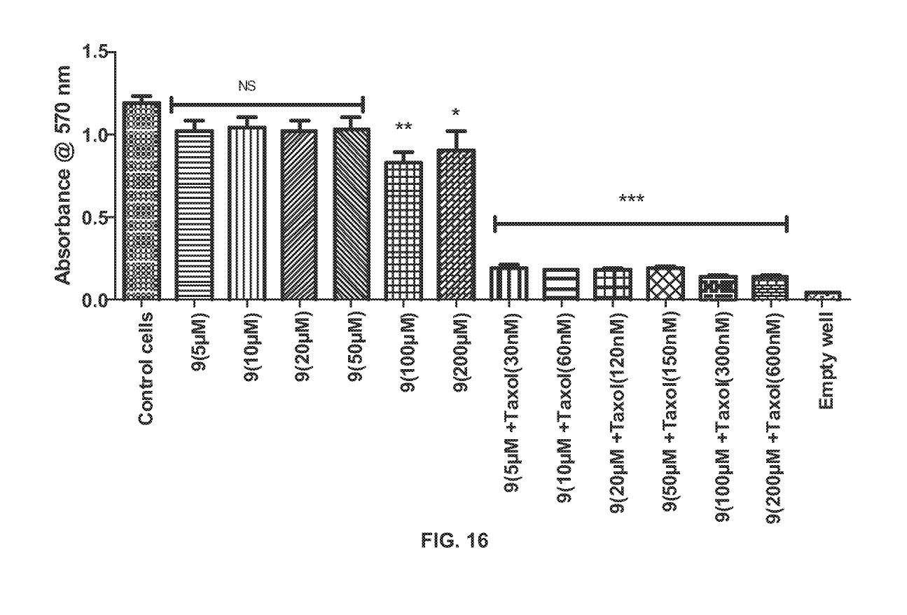

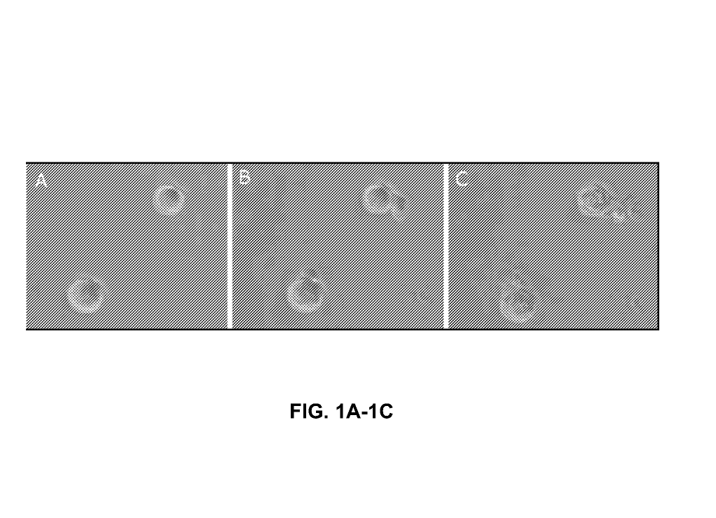

A peptoprime labeled 9R (SEQ ID NO: 199) and its scrambled peptides were synthesized and tested for bioactivity. In particular, scrambled peptide labeled 9S1 (SEQ ID NO: 200) was highly effective at killing several cancer cells including, breast and prostate cancer cells. The IC50 (concentration at which there are 50% live cells compared to untreated control) for 9S1 was 20-25 μm and killing effects were observed in as little as 2 hours. FIGS. 1A-1C show different time elapsed images of HUT-102 leukemic cells after incubation with 100 μm of 9R. FIG. 1A is the image right after incubation. In this figure, extrusion of the vesicle/bleb can be seen on the cell (bottom left). FIG. 1B is the image, 35 minutes after incubation. FIG. 1C is the image, 60 minutes after incubation. In this figure, an explosion of the vesicle/bleb structure can be seen. What remains are cellular debris and a picnotic structure. Parts of the parent cell remain associated.

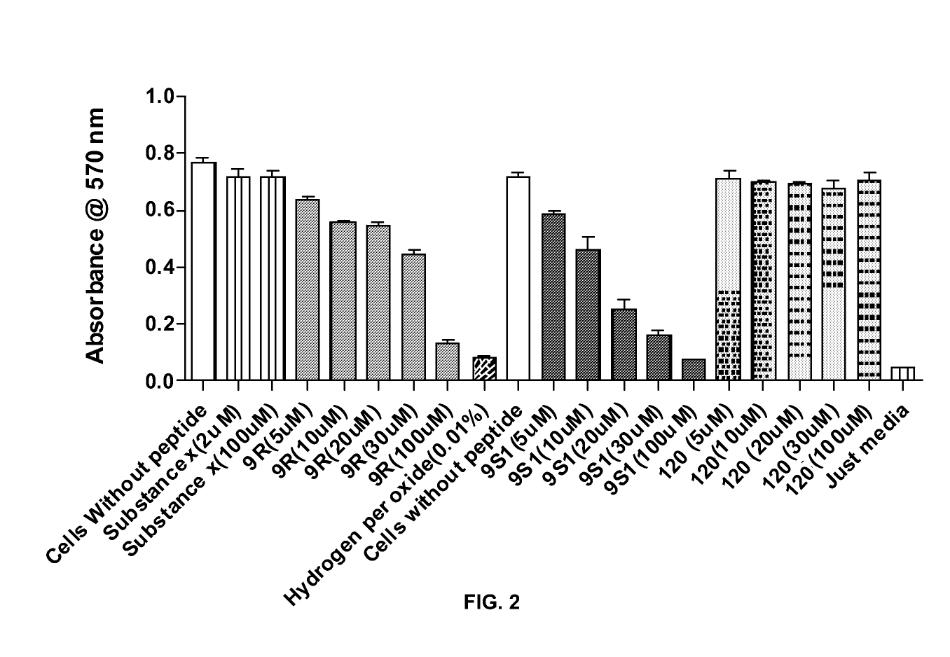

LnCaP cells were treated with peptides 9R, 9S1, 120 (SEQ ID NO: 120) and a vehicle control. LnCaP cells were treated with the peptides for 48 hours, and incubated with MTT for 4 hours. For background level of absorbance, media alone was measured on the plate. The peroxide, 9R, and 9S1 treated cells showed marked decrease in MTT absorbance value compared with control treatment cells. FIG. 2 summarizes the MTT results. Peptide 120 showed no effect on LnCap cells and may be used as a control peptide that does not exhibit cytotoxicity with these cells. Several concentrations of peptides were used to determine the IC50 of these peptides.

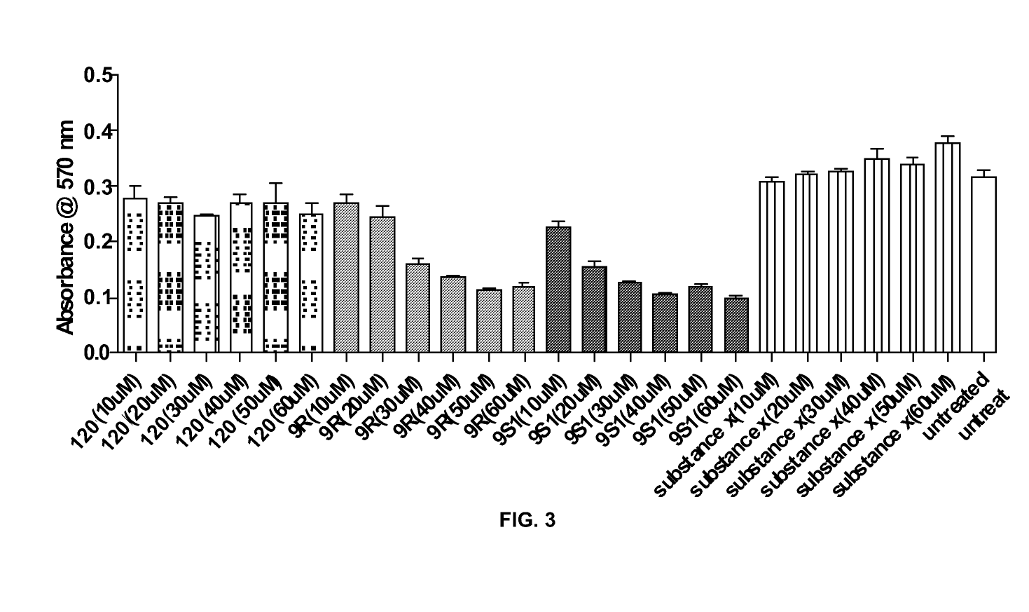

Breast cancer cells (MDA-MB-231) were also treated with peptides 9R, 9S1, and 120. Cells were treated with peptides for 1 hour and followed by MTT for 4 hours. To determine the background level of absorbance, media alone was added to several wells on the plate. A summary of MTT assay of MDA-MB-231 cells treated with peptide 9R, 9S1, and 120 is shown in FIG. 3. In this experiment, the carbohydrate was used as the vehicle control. The 9R and 9S1 treated cells showed marked decrease in MTT absorbance values compared to the control treatments. Peptide 120 showed no effect on MDA-MB-231 cells.

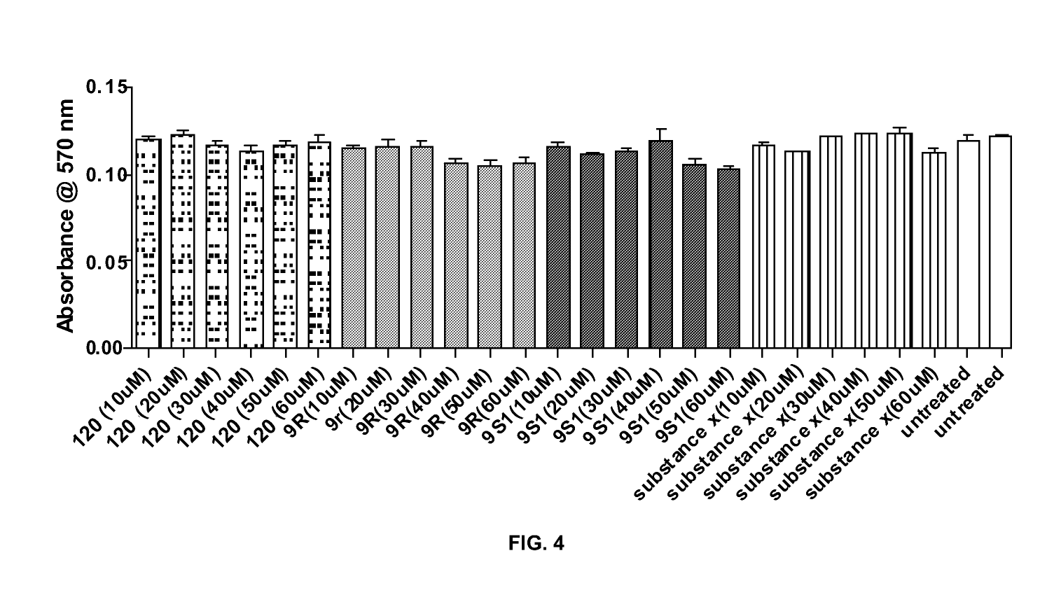

Normal (non-cancer) human lung fibroblast cells (WI-38) were also treated with the 9R, 9S1, and 120 peptides. WI-38 cells were treated with the peptides for 1 hour and followed by MTT for 4 hours. A summary of the MTT results are shown in FIG. 4. Although the treated cells show no decrease in MTT absorbance, this may have been due to the cells being plated at lower concentration, and may not have been actively dividing. Peptide 120 showed no effect on these normal fibroblast cells.

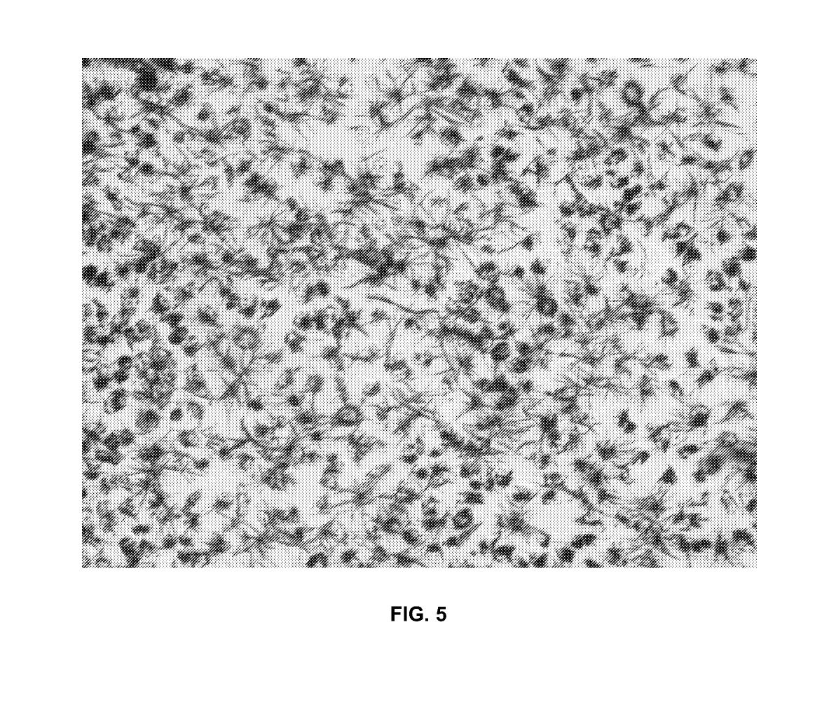

FIG. 5 shows the result of reduced formazan crystals that form around metabolically active LnCap cells.

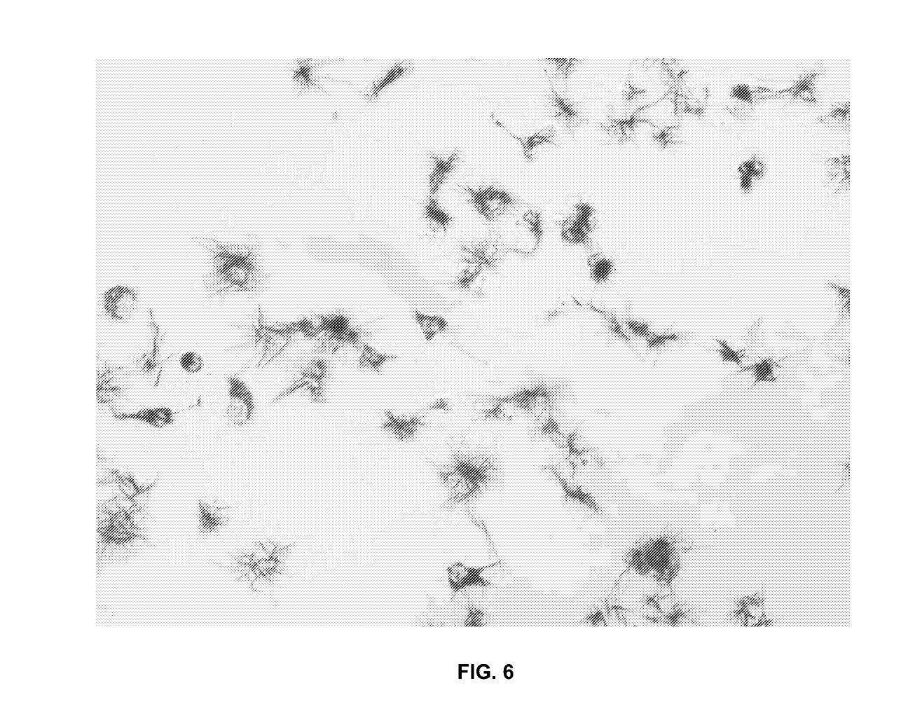

When cells were treated with a cytotoxic peptoprime, the MTT absorbance decreases, indicating fewer purple formazan crystals forming. Under the microscope (200×), fewer cells were metabolically active, and thus producing crystals. FIG. 6 illustrates this with LnCap cells treated with 30 μM peptide 9R for 48 hrs. FIG. 7 shows LnCap cells treated with 30 μM of peptide 9S1. The cells were clear (colorless), which indicate that the cells were not metabolizing. The 30 μM concentration value of the peptides is close to the IC₅₀ of peptide 9S1, and somewhat below IC₅₀ for peptide 9R (previous results show the IC₅₀ for 9R is around 50 μM).

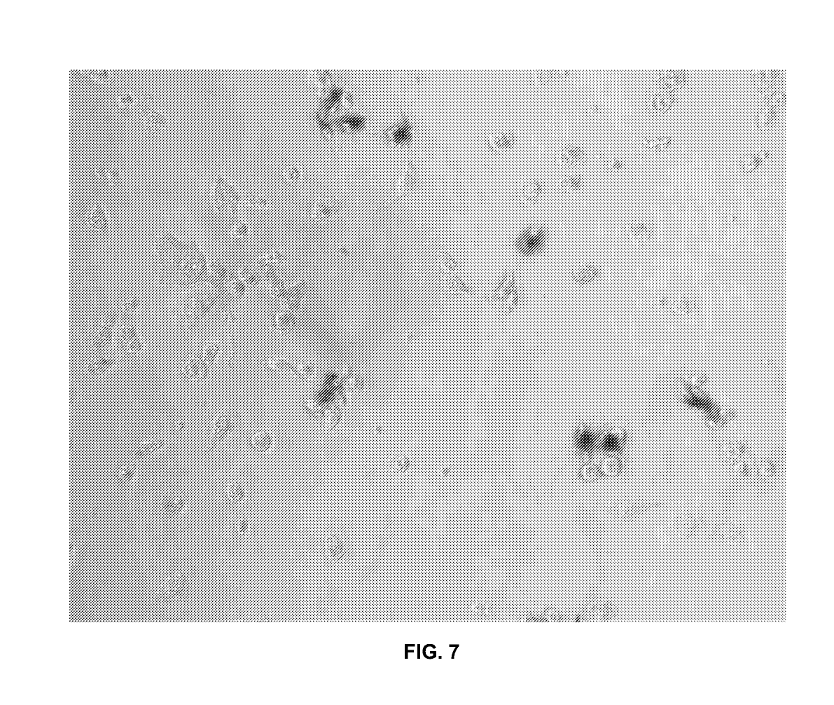

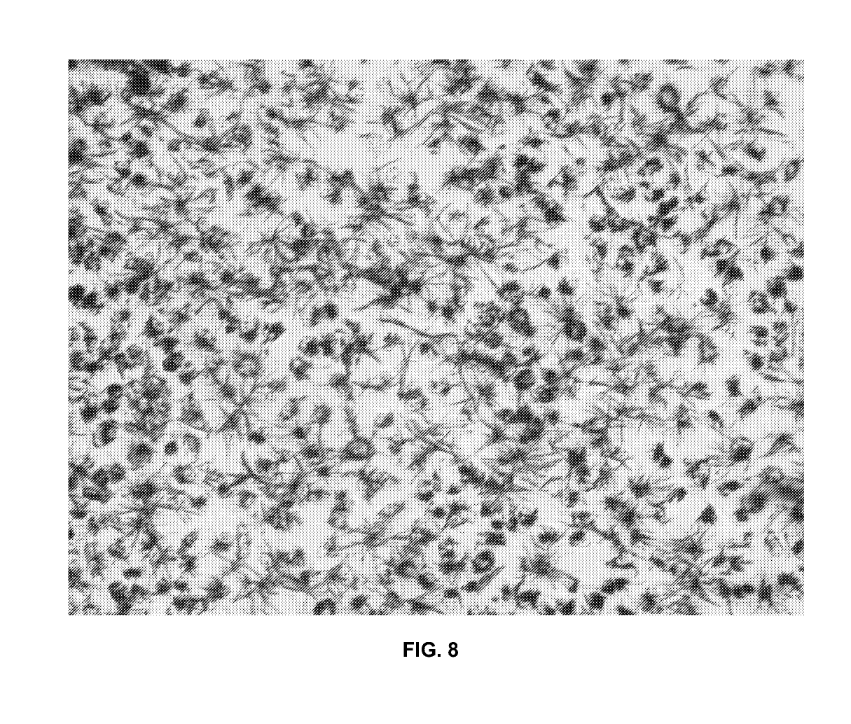

FIG. 8 shows a field full of metabolizing cells, similar to the image of healthy, untreated cells. These cells were treated with an identical concentration (30 μM) of peptoprime peptide 120.

### Example 3

The effect of peptoprime 9S1 on mitochondrial health was evaluated with confocal microscopy.

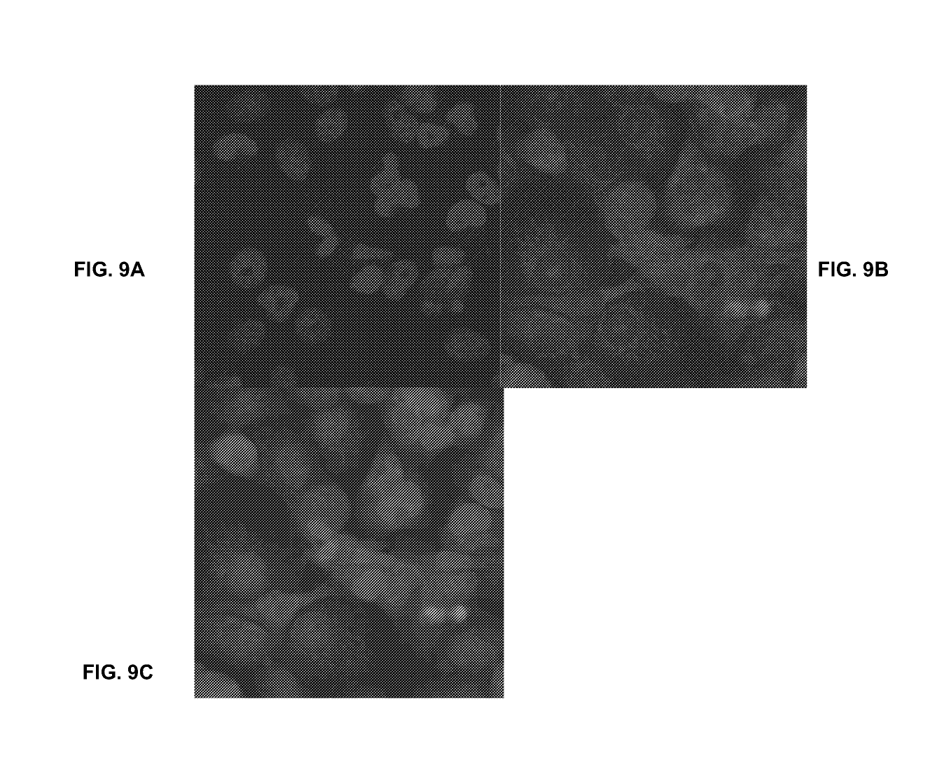

The HCS MITOCHONDRIAL HEALTH KIT™ (available from Invitrogen) includes a dye that is taken up by healthy, actively respiring mitochondria. The uptake of this dye can be visualized with fluorescent confocal microscopy. FIGS. 9A-9C show control LnCaP cells treated only with the vehicle control (i.e., no peptide). The Hoechst dye stained nuclei of cells, whether they were alive or dead (Excitation/Emission maxima—Hoechst 33342: 350/461 nm). FIG. 9A is a panel showing all nuclei stained with Hoechst dye. FIG. 9B is a panel showing healthy mitochondria stained with MitoHealth dye. FIG. 9C is a panel showing the merged images of 9A and 9B. The mitochondrial health dye incorporated into the cells depends on mitochondrial membrane potential (excitation/emission maxima—MitoHealth stain: 550/580 nm). The HCS MITOCHONDRIAL HEALTH KIT™ (Invitrogen) was developed for simultaneous quantitative measurements of two cell health parameters in the same cell: mitotoxicity and cytotoxicity. The MitoHealth stain accumulated in mitochondria in live cells proportional to the mitochondrial membrane potential.

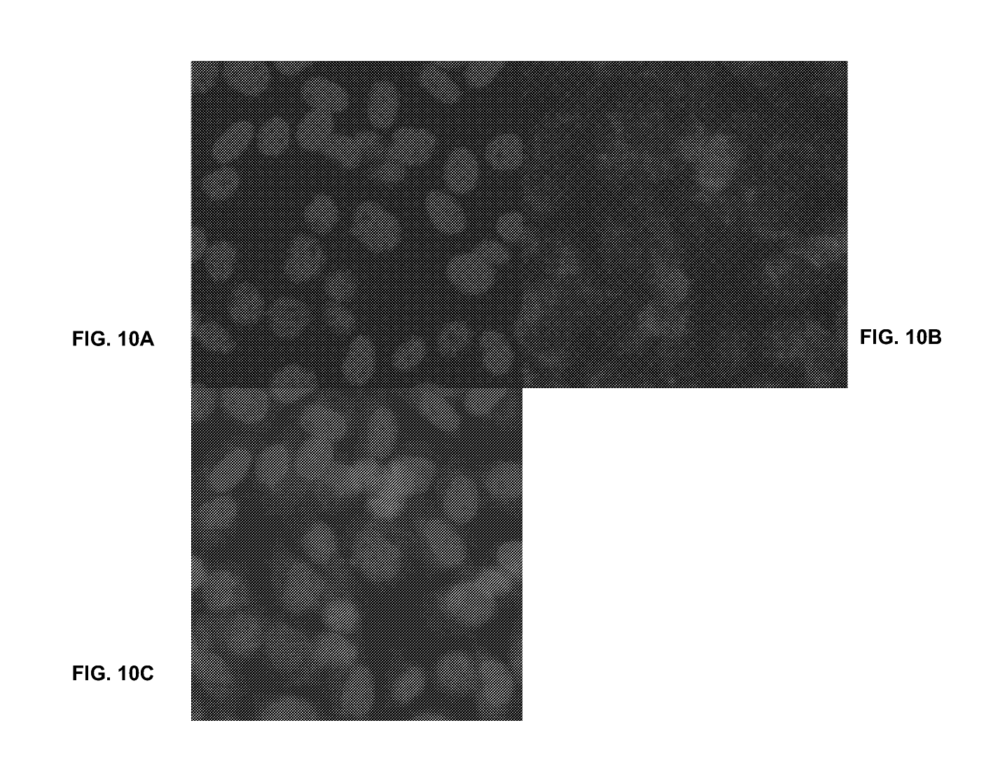

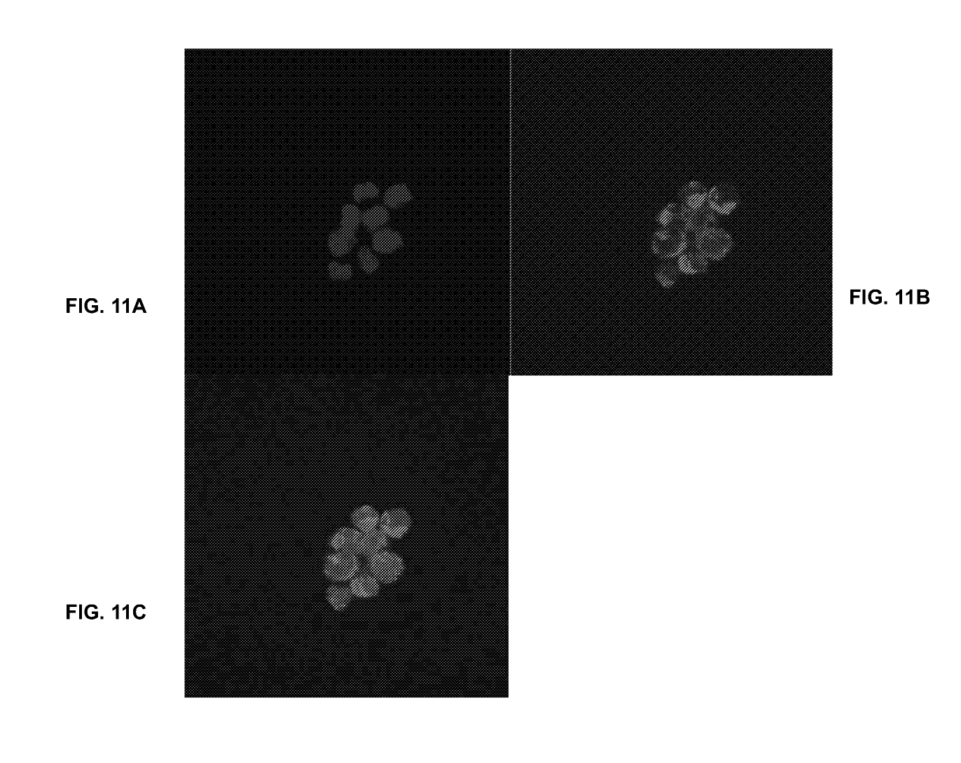

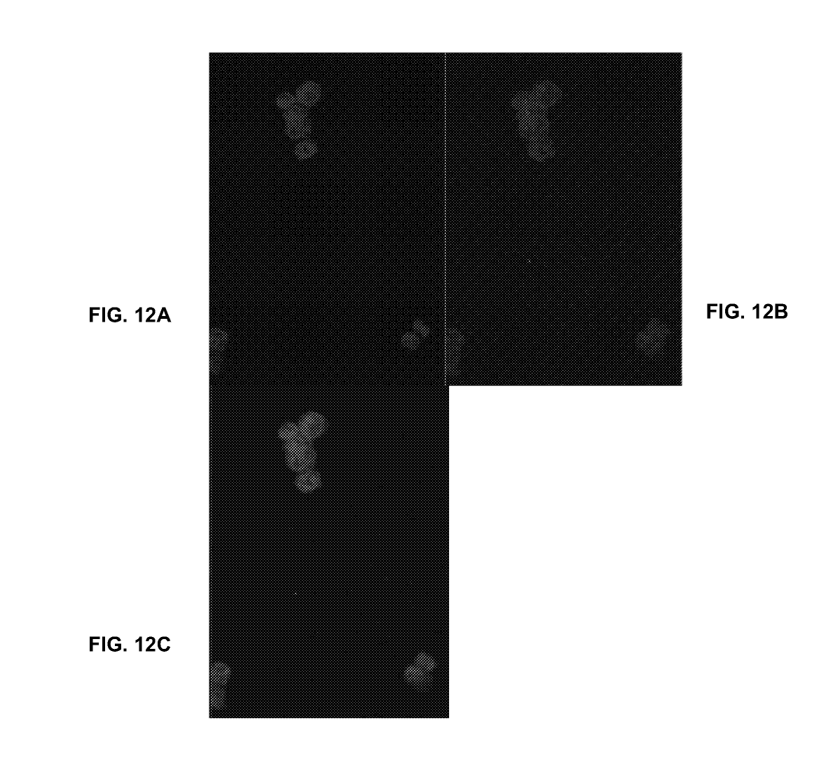

When LnCaP cells were incubated with 100 μM of peptide 951 for 1 hour, a decrease in overall fluorescence was observed (FIGS. 10A-10C). The mitochondria themselves were also not as distinctive (diffusely staining). The same results were seen for HUT-102 cells, in 9S1 treated versus vehicle-control treated cells (FIGS. 11A-11C). A clear decrease in mitochondrial fluorescence was seen (FIGS. 11 and 12).

FIG. 12 shows a 64× amplified image of HUT-102 cells treated with nullomer peptide 9S1 at 100 μM concentration.

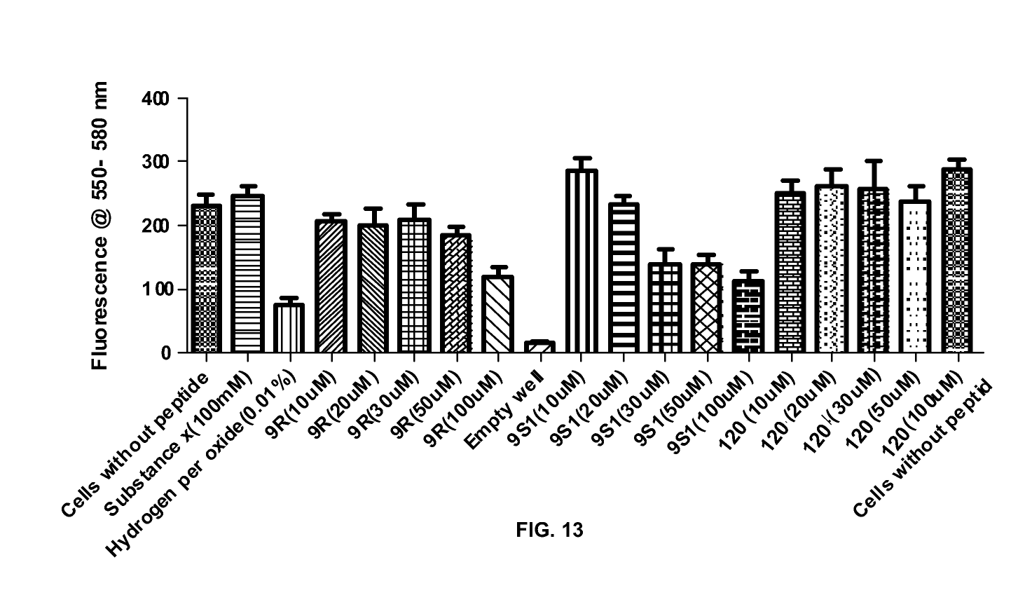

FIG. 13 shows the results of the mitochondrial health assay as read by the plate reader. A range of peptide concentrations was applied to the LnCap cells. 9R showed an IC50 (50% inhibitory concentration) of 100 μM whereas peptide 9S1 showed IC50 at 30 uM. Peptide 120 did not have any effect on the LnCap cells' mitochondrial membrane potential. The cells were treated for 24 hrs. This data also complement the confocal imaging experiments, where decreased fluorescence was seen in peptide 9S1-treated cells.

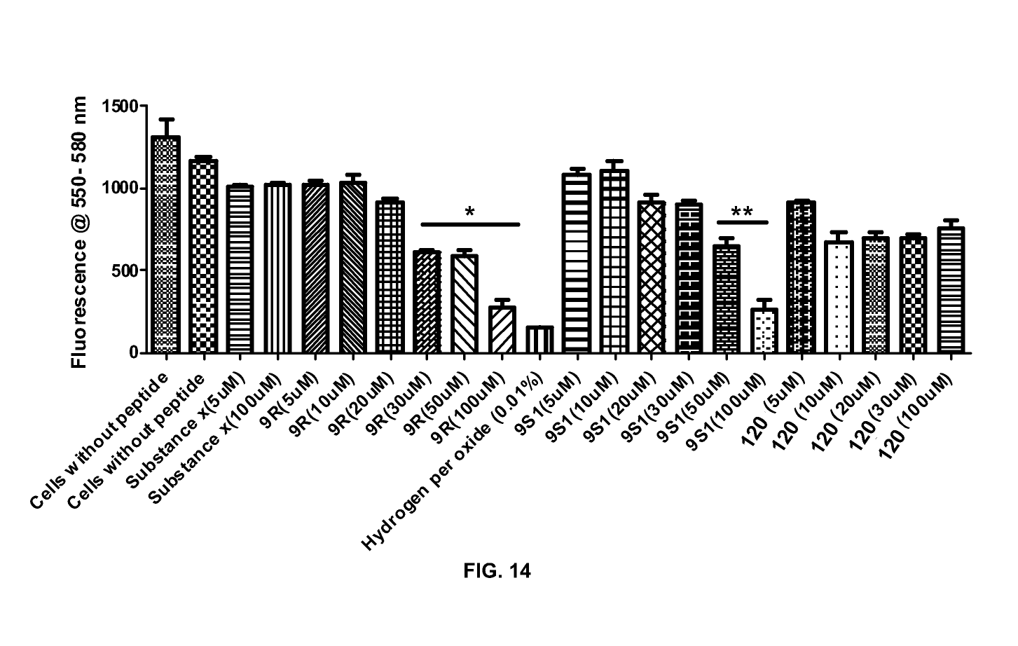

FIG. 14 shows the results of the mitochondrial health assay, run on a BioTek SYNERGYMX plate reader. A range of peptide concentrations was used to treat LnCap cells. Peptide 120 did not have any measurable effect on the mitochondrial health of the LnCap cells. The cells were incubated with peptide for 24 hrs. This data complements the data from confocal imaging of treated and untreated cells, and shows a significant difference between vehicle-control treated cells (carbohydrate, both 5 and 100 μM) and cells treated with 9R above 30 μM (repeated measures of ANOVA with Bonferroni's Multiple Comparison Test, p<0.01).

### Example 4

The 9S1 peptide also showed rapid increases in the production of reactive oxygen species (ROS) in mitochondria of prostate cancer cells (LnCap) in culture.

The MITOSOX™ Red reagent was used to detect ROS. MITOSOX™ Red mitochondrial superoxide indicator is a novel fluorogenic dye for highly selective detection of superoxide in the mitochondria of live cells. MitoSOX™ Red reagent is live-cell permeant and rapidly and selectively targets the mitochondria. Once in the mitochondria, MitoSOX™ Red reagent is oxidized by superoxide and exhibits red fluorescence. Mitochondrial superoxide is typically generated as a byproduct of oxidative phosphorylation. MitoSOX™ Red reagent is readily oxidized by superoxide but not by other ROS- or reactive nitrogen species (RNS)-generating systems.

The MITOSOX™ Red reagent kit was used to detect superoxide generated in the mitochondria of live cells, and identify agents that modulate oxidative stress in various pathologic cells. In an otherwise tightly coupled electron transport chain, approximately 1-3% of mitochondrial oxygen consumed was incompletely reduced. It is believed that the “leaky” electrons can quickly interact with molecular oxygen to form the superoxide anion, the predominant reactive oxygen species (ROS) in mitochondria.

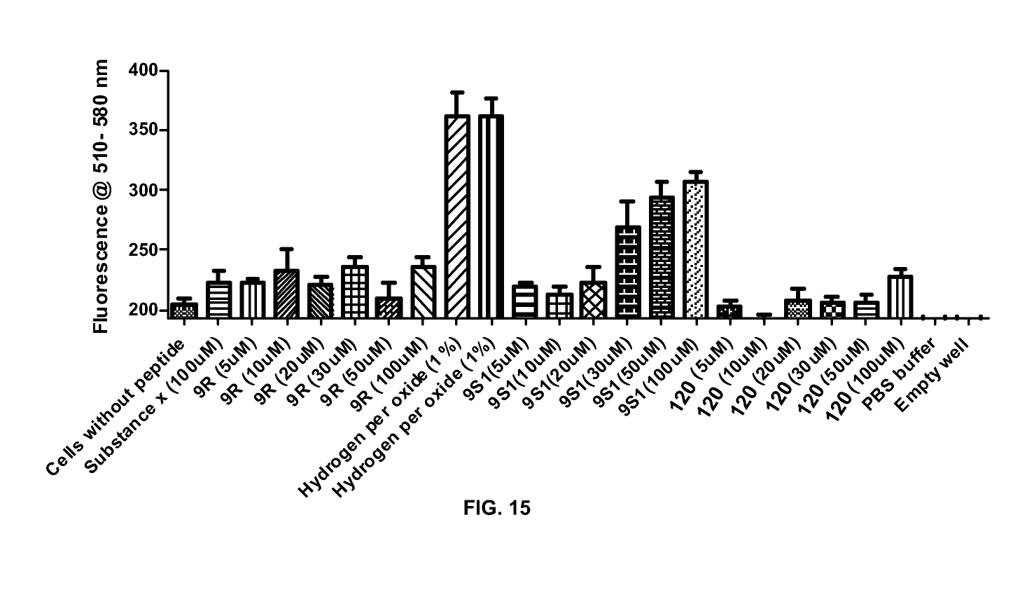

FIG. 15 shows that peptide 9S1-treated cells have higher levels of ROS production. At 50 and 100 μM 9S1, there was significant increase in ROS production, compared to peptide 9R and 120 treated cells (one way ANOVA, Bonferroni correction, p<0.01). The level of ROS production was also dose dependent. Non-treated cells and peptide solvent did not produce significant amounts of ROS. Hydrogen peroxide was used as a positive control.

### Example 5

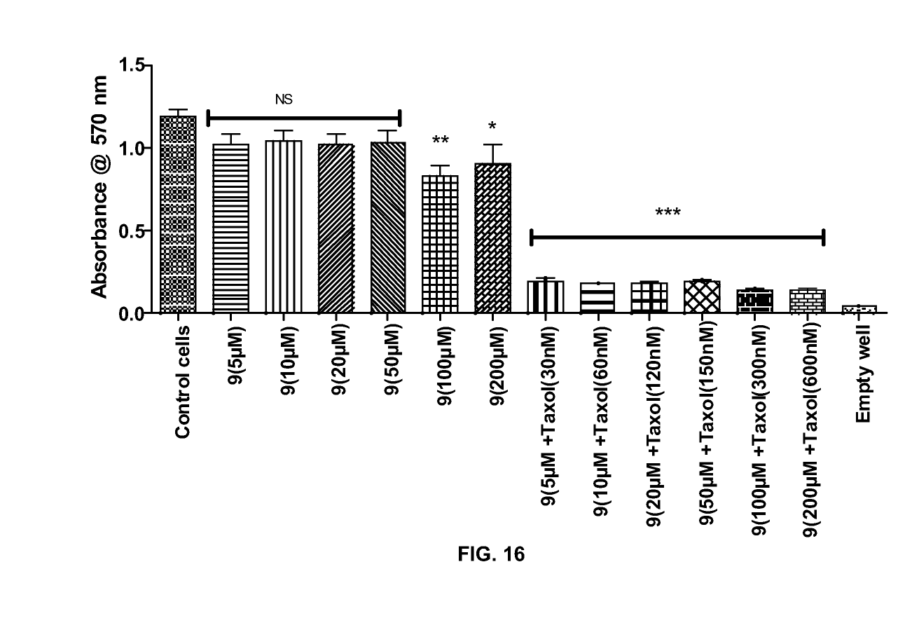

The 9R peptide (SEQ ID NO: 200; labeled “9” in FIG. 16) was tested for its ability to reverse paclitaxel (TAXOL™) resistance in paclitaxel-resistant LnCap cells. FIG. 16 shows the results of MTT assay used to show the sensitivity of paclitaxel-resistant LnCap cells to paclitaxel.

Paclitaxel-resistant LnCaP cells were treated with the peptides for 120 hours, and incubated with MTT for 4 hours. The paclitaxel-resistant LnCap cells were resistant to paclitaxel even at 600 μM concentration (data not shown). Referring to FIG. 16, the paclitaxel-resistant LnCap cells were resistant to 9R at the 100 μM level (labeled **). However, the combination of paclitaxel (at little as 30 nM) and 9R (at little as 5 μM) killed the paclitaxel-resistant LnCap cells (labeled ***).

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

1. A method comprising:
a) storing, in a memory or storage of a computing device, a set of at least one peptide sequence of fixed length;
b) searching, by the computing device, for a peptide sequence from the set within at least one database comprising naturally-occurring amino acid sequences; and
c) classifying, by the number of appearances in the database, the peptide sequence.

2. The method of claim 1 wherein the peptide sequence is classified as a common peptide, a rare peptide, a nullopep, or a peptoprime.

3. The method of claim 1 wherein the set comprises all possible peptide sequences of the fixed length.

4. The method of claim 1 wherein b and c are repeated until all peptide sequences within the set have been searched in the database.

5. The method of claim 1 further comprising:
d) re-storing, in the memory or storage of the computing device, the set of peptide sequences wherein the fixed length is increased; and
e) repeating b), c), and d).

6. The method of claim 1 further comprising: repeating a), b), and c) after the database has been updated.

7. The method of claim 1 wherein the fixed length is between about 2 to about 20 amino acids.

8. The method of claim 2 further comprising: assaying the rare peptide, the nullopep, the peptoprime, or scrambled sequences thereof for biological activity.

9. A method comprising:
a) generating, by a computing device, a set of at least one peptide sequence of fixed length;
b) searching, by the computing device, for a peptide sequence from the set within at least one database comprising naturally-occurring amino acid sequences;
c) classifying, by the number of appearances in the database, the peptide sequence as being one of: a rare peptide or an absent peptide; and
d) contacting the rare peptide, the absent peptide, or a scrambled sequence thereof with an organism, a cell, or a virus.

10. The method of claim 9 wherein the absent peptide is a nullopep or a peptoprime.

11. The method of claim 9 wherein the cell has a resistance to a drug.

12. The method of claim 9 wherein the cell has a pathological disease.

13. The method of claim 12 wherein the pathological disease comprises a cancer selected from the group consisting of: breast cancer, prostate cancer, leukemia, melanoma, and any combination thereof.

14. The method of claim 9 wherein the peptide is modified by bonding or complexing with a composition selected from the group consisting of: a second peptide, an antibody, an antigen, a fluorescent molecule, a carbohydrate, a nucleotide, a lipid, an arginine, a nucleic acid, a metal, a glass, a silicate, a mineral, a radioisotope, a pharmaceutical compound, a growth factor, a primer, a biomarker, a nucleic acid, a dye, an adjuvant, a natural or synthetic polymer, and any combination thereof.

15. The method of claim 9 wherein the peptide has a sequence selected from the group consisting of SEQ ID NOS: 1-201.

16. A synthetic or isolated peptide having a sequence selected from the group consisting of SEQ ID NOS: 1-201.

17. The synthetic or isolated peptide of claim 16 is modified by bonding or complexing with a composition selected from the group consisting of: a second peptide, an antibody, an antigen, a fluorescent molecule, a carbohydrate, a nucleotide, a lipid, an arginine, a nucleic acid, a metal, a glass, a silicate, a mineral, a radioisotope, a pharmaceutical compound, a growth factor, a primer, a biomarker, a nucleic acid, a dye, an adjuvant, a natural or synthetic polymer, and any combination thereof.

18. The synthetic or isolated peptide of claim 17 wherein the modification is an addition of 2 or more arginines to the N-terminal or C-terminal end of the peptide.

19. The synthetic or isolated peptide of claim 16 further comprising a modification selected from the group consisting of: enhancing the solubility of the peptide, enhancing the binding of the peptide to a cell surface protein, enhancing activation of an immunological response, enhancing mitochondrial impairment, and any combination thereof.

20. The synthetic or isolated peptide of claim 19 wherein the immunological response comprises an activation of a composition selected from the group consisting of: activation of a B cell, a B Cell progenitor, a T cell, a T cell progenitor, a mast cell, a langerhans cell, a dendritic cell, a natural killer cell, a granulocyte-monocyte progenitor, a monocyte, a neutrophil, an eosinophil, a basophil, a magakaryocyte, a platelet, an erythrocyte, an erythroid progenitor cell, and any combination thereof.
